(19) United States
(12) Patent Application Publication
(10) Pub. No.: US 2024/0252594 A1
(43) Pub. Date: Aug. 1, 2024

(54) COMBINATION THERAPY FOR NEURODEGENERATIVE DISEASES

(71) Applicant: ALETA NEUROSCIENCE, LLC, MARLTON, NJ (US)

(72) Inventor: Alexander SHTILBANS, Englewood Cliffs, NJ (US)

(21) Appl. No.: 18/610,040

(22) Filed: Mar. 19, 2024

Related U.S. Application Data
(63) Continuation of application No. PCT/IB2022/058850, filed on Sep. 19, 2022.
(60) Provisional application No. 63/261,379, filed on Sep. 20, 2021.
(60) Provisional application No. 63/264,035, filed on Nov. 13, 2021.

(51) Int. Cl. A61K 38/26; A61K 31/192; A61K 31/4412; A61K 31/506; A61K 31/575; A61P 25/28

(52) U.S. Cl. CPC A61K 38/26; A61K 31/192; A61K 31/4412; A61K 31/506; A61K 31/575; A61P 25/28

(57) ABSTRACT

This invention provides compositions and methods for treating or preventing neurodegenerative disorders with combinations of at least two drugs from two or more classes of pharmacological activity. The subject neurodegenerative disorders are associated with misfolding of tau proteins, amyloid, alpha-synuclein, superoxide dismutase 1 (SOD1), Tar DNA binding protein-43 (TDP43), Ubiquilin-2, p62, valosin-containing protein (VCP), huntingtin protein (mHtt) and dipeptide repeat (DPR) proteins. The pharmacological classes include a chemical chaperone class of drugs including bile acids, a Heat Shock Proteins (HSP) co-inducer class of drugs, a glucagon-like-peptide-1 agonist (GLP-1) class of drugs, an iron chelator class of drugs, and a cluster-Abelson (c-Abl) tyrosine kinase inhibitor class of drugs.

### FIELD OF THE INVENTION

Combinations of drugs from at least two pharmacological classes are provided as compositions and methods for the treatment neurodegenerative diseases associated with misfolding of key neuroproteins, neuroinflammation and mitochondrial dysfunction by interrupting multiple pathways leading to neurodegeneration.

### BACKGROUND

Neurodegenerative disorders (ND) are devastating diseases characterized by progressive and irreversible neuronal dysfunction and death(1). The pathophysiological mechanisms of these diseases are diverse and involve distinct subgroups of neurons in specific areas of the brain(2). These diseases include: Alzheimer's disease (AD), Parkinson's disease (PD), Amyotrophic Lateral Sclerosis (ALS), Frontotemporal Dementia (FTD), Corticobasal Degeneration (CBD), Progressive Supranuclear Palsy (PSP) and Huntington's disease (HD). These diseases affect many people globally causing severe distress for patients and caregivers, and also result in a large socioeconomic burden(3). There is a critical need to develop new and more efficient therapies to combat these prevalent disorders. In all these diseases, misfolding of key neuroproteins in the brain leads to abnormal aggregation of neuroproteins and pathology.

In AD, deposition and aggregation of misfolded intracellular tau protein-aggregates in the neurofibrillary tangles and extracellular AB aggregates in the senile plaques are the key pathological hallmarks(4). Other tauopathies such as Progressive Supranuclear Palsy (PSP), Frontotemporal dementia (FTD) and Corticobasal Degeneration (CBD) also involve the accumulation of misfolded tau protein. Misfolded proteins linked to ALS include superoxide dismutase 1 (SOD1), Tar DNA binding protein-43 (TDP43), Ubiquilin-2, p62, valosin-containing protein (VCP), and dipeptide repeat (DPR) proteins(5). In PD, misfolding and aggregation of α-synuclein (the main component of Lewy bodies), is the main pathological characteristic. Like α-synuclein, parkin (a proteasome-associated ubiquitin ligase) is also prone to misfolding and plays a key role in the pathogenesis of PD(6). HD is a consequence of mutation in the huntingtin protein (Htt) encoding gene, which leads to the expansion of CAG repeats encoding for a stretch of polyglutamine (polyQ). The polyQ stretch is pathogenic when it contains more than 35 glutamines. The resultant mutant huntingtin protein (mHtt) is prone to misfolding and aggregation(7). The misfolding and aggregation of different proteins into irregular, toxic species results in neurotoxicity in these neurodegenerative diseases(8),(9).

All of these disorders are described by the accumulation, in the form of high-ordered insoluble fibrils, of one or more abnormal proteins within intra- or extracellular aggregates. The identity of the underlying protein dictates which neurons are affected by the disease and thus, the clinical manifestations of each disease(10). Many studies have indicated that protein misfolding and aggregation, leading to ER stress, are central factors of pathogenicity in ND(7).

There is no obvious sequence or structural homology among the proteins involved in various neurodegenerative diseases but there is a significant structural rearrangement in all cases between the monomeric native protein and the aggregated material. Another hallmark of these neurodegenerative diseases is neuroinflammation in the brain that sets in at some point, directly or indirectly related to the aggregated misfolded proteins, and further contributes to neurodegeneration. Additional common features in the ND are mitochondrial dysfunction leading to energy depletion, and disruption of brain iron homeostasis leading to abnormal iron accumulation in parts of the brain mostly involved in each of the above-mentioned ND which results in reactive oxygen species (ROS) formation further contributing to neurodegeneration.

Several classes of medications have been tried individually in the past to slow progression of these neurodegenerative disorders but have not shown clinical efficacy, likely because most prior attempts to treat neurodegeneration have only targeted one specific part of the above-described complex neurodegenerative process (FIG. 1), which was not enough to show meaningful clinical effect. Moreover, the diversity and individual predominance of different parts of neurodegenerative chain in each individual patient made it impossible to discern potential improvement in the heterogeneous groups of patients evaluated in those studies. Indeed, there are four major common processes leading to the above neurodegenerative disorders: accumulation of a misfolded protein, mitochondrial dysfunction, iron accumulation and neuroinflammation that could be occurring at different rates in different affected individuals. No studies to our knowledge ever targeted all of these processes simultaneously by combining drugs aimed at each of them which we propose to do using the below classes of medications in different combinations (FIG. 1).

### SUMMARY OF THE INVENTION

This invention provides compositions and methods for treating or preventing neurodegenerative disorders, which have resisted therapeutic interventions to date. The inventive compositions are combinations of at least two drugs from two or more classes of pharmacological activity as described herein.

In an embodiment, a composition and method is provided for use in treating or preventing a neurodegenerative disorder associated with misfolding of proteins including tau proteins, amyloid, alpha-synuclein, superoxide dismutase 1 (SOD1), Tar DNA binding protein-43 (TDP43), Ubiquilin-2, p62, valosin-containing protein (VCP), huntingtin protein (mHtt) and dipeptide repeat (DPR) proteins. The composition may include a combination of at least two drugs selected from distinct pharmacological classes of drugs, wherein the distinct classes of drugs include a chemical chaperone class of drugs, a Heat Shock Proteins (HSP) co-inducer class of drugs, a glucagon-like-peptide-1 agonist (GLP-1) class of drugs, an iron chelator class of drugs, and a cluster-Abelson (c-Abl) tyrosine kinase inhibitor class of drugs. In an embodiment, the composition includes a combination of at least three drugs selected from two of the classes, or at least three drugs selected from three of the distinct classes.

In a further aspect, the composition may include the following specific embodiments:
a. the chemical chaperone class of drugs includes sodium phenylbutyrate (PBA) and a bile acid including one or more of tauroursodeoxycholic acid (TUDCA), ursodeoxycholic acid (UDCA) and deoxycholic acid (DCA);
b. HSP co-inducer class of drugs comprises one or more of arimoclomol and bimoclomol;
c. wherein the GLP-1 class of drugs includes one or more of Exenatide, ORMD-0901, dulaglutide, semaglutide, liraglutide, lixisenatide, and NLY01;
d. wherein the iron chelator class of drugs includes a drug selected from deferiprone (DFP), deferoxamine (DFO), desferrioxamine, deferasirox, clioquinol, tetrahydrosalen, 5,7-Dichloro-2-[(dimethylamino)methyl]quinolin-8-ol (PBT2), (N,N,N,N-Tetrakis(2-pyridylmethyl)-ethylenedi-amine) (TPEN), 1,10-phenanthroline (PHEN), 1,2-hydroxypyridinone (1,2-HOPO), clioquinol; 5-[N-methyl-N-propargylaminomethyl]-8-hydroxyquinoline dihydrochloride (M30); M31; M32; -[4-(2-hydroxyethyl)piperazine-1-ylmethyl]-quinoline-8-ol] (VK28), HLA16, HLA20, M32, M10, SIH-B, BSIH, pyridoxal isonicotinoyl hydrazine (PIH); 2-pyridylcarboxaldehyde isonicotinoyl hydrazine (PCIH), H2NPH, and H2PPH;
e. wherein the c-Abl tyrosine kinase inhibitor class of drugs includes a drug selected from nilotinib radotinib, vodobatinib (K0706), bafetinib, imatinib, dasatinib, bosutinib, ponatinib, rebastinib, tozasertib, danusertib and IkT-148009.

The neurogenerative disorder may be selected from Alzheimer's disease (AD), Parkinson's disease (PD), Amyotrophic Lateral Sclerosis (ALS), Huntington's disease (HD), Progressive Supranuclear Palsy (PSP), Frontotemporal dementia (FTD) and Corticobasal Degeneration (CBD).

Some specific embodiments include combinations of the following drugs: sodium phenylbutyrate (PBA) and exenatide (EXD); PBA, tauroursodeoxycholic acid (TUDCA), and EXD; nilotinib (NL) and TUDCA; EXD and TUDCA; EXD, NL, and TUDCA; PBA, EXD, and deferiprone (DFP); PBA, NL, and TUDCA; EXD and NL; PBA, EXD, and NL; PBA, EXD, TUDCA, and DFP; PBA, arimoclomol (ARM), and TUDCA; and EXD and ARM.

### DESCRIPTION OF THE DRAWINGS

FIG. 1 Shows multiple processes leading to neurodegeneration. Numbers in parentheses refer to a drug name/class that affects the numbered process: (1) chemical chaperone class of drugs, (2) a bile acids, (3) a glucagon-like-peptide-1 agonist (GLP-1) class of drugs; (4) an iron chelator class of drugs; (5) a cluster-Abelson (c-Abl) tyrosine kinase inhibitor class of drugs; and (6) a Heat Shock Proteins (HSP) co-inducer class of drugs.

FIG. 2 is a plot of neurite length (mm/mm²) summarizing data for controls and combinations 8, 10,12. Statistical analysis for FIGS. 2-11: one-way ANOVA followed by Bonferroni's multiple comparisons test in case of significant effect (*=p<0.05; **=p<0.01; ***=p<0.001). Key to notations in FIGS. 2-11: (a): compared with Control/NT. (b): compared with Control/MPP+. NT: Not Treated; PCI (Pan Caspase Inhibitor): positive control.

FIG. 3 is a plot of neurite branching (mm²) summarizing data for controls and combinations 8, 10, 12.

FIG. 4 is bar plot of neurite length (mm/mm²) summarizing data for controls and combinations 22, 24.

FIG. 5 is a bar plot of neurite branching (mm²) summarizing data for controls and combinations 22, 24.

FIG. 6 is bar plot of neurite length (mm/mm²) summarizing data for controls and combinations 24, 38, 40. For FIGS. 6-11, PCI is a Pan Caspase Inhibitor positive control.

FIG. 7 is a bar plot of neurite branching (mm²) summarizing data for controls and combinations 24, 38, 40.

FIG. 8 is a plot of neurite length (mm/mm²) summarizing data for controls and combinations 10, 12, 51-53.

FIG. 9 is a plot of neurite branching (mm²) summarizing data for controls and combinations 10, 12, 51-53.

FIG. 10 is a bar plot summarizing the data for cytolysis (%) of controls plus experimental combinations 10, 12, 51-53.

FIG. 11 is a bar plot summarizing the data for cytolysis (%) of controls plus experimental combinations 24, 38, 40.

FIG. 12 shows data from the α-synuclein triplication cell line experiment, showing that the combination of EXD and TUDCA has significant activity compared to the individual drugs and other combinations studied.

FIGS. 13-15 are bar plots of the data in Tables 13.1, 13.2, and 13.3 for the neuroinflammation in microglia experiments. FIG. 13 shows the inhibition of IL-6 mediated inflammation in microglial cells. FIG. 14 shows inhibition of TNF-α mediated neuroinflammation in microglial cells. FIG. 15 shows inhibition of CXCL1/GROα mediated neuroinflammation in microglial cells.

### DETAILED DESCRIPTION

Disclosed herein are combinations of drugs as compositions and methods designed to treat or prevent neurogenerative disorders related to misfolding of key proteins, neuroinflammation and mitochondrial dysfunction. Key proteins pertinent to this invention include tau proteins, superoxide dismutase 1 (SOD1), Tar DNA binding protein-43 (TDP43), Ubiquilin-2, p62, valosin-containing protein (VCP), and dipeptide repeat (DPR) proteins, alpha-synuclein and huntingtin protein (mHtt). The neurodegenerative disorders include Alzheimer's disease (AD), Parkinson's disease (PD), Amyotrophic Lateral Sclerosis (ALS), Huntington disease (HD), Corticobasal degeneration (CBD), progressive supranuclear palsy (PSP), and Frontotemporal Dementia (FTD).

In an embodiment, a composition and method is provided for treating or preventing a neurodegenerative disorder, by the administration of a combination of two or more drugs selected from a chemical chaperone class of drugs (including bile acids), a Heat Shock Protein (HSP) co-inducer class of drugs, a glucagon-like-peptide-1 agonist (GLP-1) class of drugs, an iron chelator class of drugs, and a cluster-Abelson (c-Abl) tyrosine kinase inhibitor class of drugs. The combination of drugs may include two or more drugs selected from at least two of the distinct pharmacological classes on this list or three or more drugs selected from at least three of the pharmacological classes on this list.

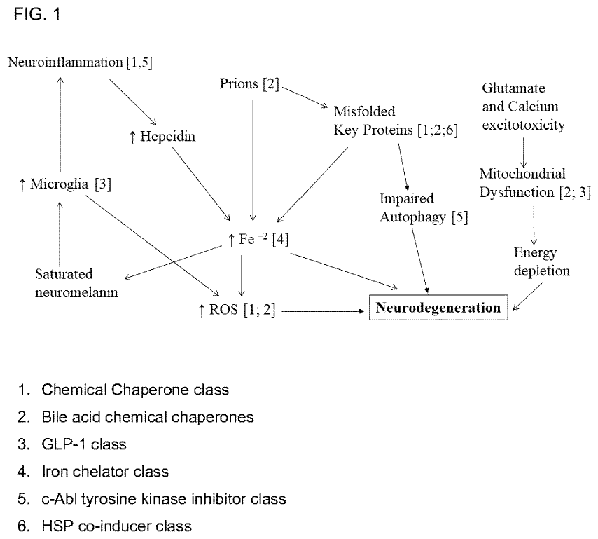

A number of specific processes are implicated in neurodegenerative diseases. FIG. 1 is a diagram showing relationships believed to lead to neurodegeneration in disorders discussed herein. Starting from the top right of FIG. 1, overaccumulation and misfolding of key proteins in the brain coupled with impaired autophagy leads to neurodegeneration. Moreover, misfolded proteins lead to over-accumulation of intracellular iron(II) in the brain which in turn leads to formation of reactive oxygen species (ROS) and neurodegeneration(11),(12). The over-accumulated iron is removed from the brain by the natural iron chelator neuromelanin(13), (14). Once neuromelanin is saturated with iron, it causes activation of microglia which leads to formation of more ROS as well as neuroinflammation which in turn results in more brain iron accumulation closing the feedback loop(15).

A second pathway is glutamate and calcium excitotoxicity which leads to mitochondrial dysfunction, energy depletion, and neuron death. In excitotoxicity, nerve cells suffer damage or death when the levels of otherwise necessary and safe neurotransmitters such as glutamate become pathologically high, resulting in excessive stimulation of receptors. For example, when glutamate receptors such as the NMDA receptor or AMPA receptor encounter excessive levels of the excitatory neurotransmitter glutamate, significant neuronal damage may ensue. Excess glutamate allows high levels of calcium ions (Ca²⁺) to enter the cell. Highly elevated intraneuronal calcium levels are implicated in mitochondrial dysfunction and the production of reactive oxygen species leading to apoptosis.

Numbers in brackets in FIG. 1 refer to classes of drugs which inhibit that particular process. For example, activation of microglia can lead to neuroinflammation and neurodegeneration, and GLP-1 agonists (#3) inhibit activation of microglia.

Tables 1a and 1b list drug combinations proposed in this invention. These drugs have been previously studied individually for neurodegenerative disorders. This invention proposes to use various combinations of the compounds in Tables 1a and 1b, which are postulated to stop, slow down, or prevent individual parts of the neurodegenerative processes seen in PD, AD, ALS, HD, FTD, CBD, PSP which we expect can produce synergistic effects needed to show clinical efficacy in human trials.

### Chaperones

### Chemical Chaperones

Chemical chaperones are small molecule bioactive compounds that enhance the folding and/or stability of proteins. This invention includes sodium phenylbutyrate and bile acids as chemical chaperones.

Sodium 4-phenylbutyrate (sodium phenylbutyrate, PBA), is an FDA-approved therapy for reducing plasma ammonia and glutamine in urea cycle disorders. PBA has anti-inflammatory activity and reduces ROS and misfolded proteins in the brain. This drug is also a histone deacetylase inhibitor and can suppress both proinflammatory molecules and reactive oxygen species in activated glial cells in the brain(16). It also acts as a chemical chaperone and can prevent aggregation of misfolded proteins and suppress endoplasmic reticulum stress (ER stress)(17). Previous preclinical studies showed that it halted the disease progression in a chronic PD and DLBD mouse models and may be of therapeutic benefit for PD(16).

Alzheimer's disease. Diverse studies have demonstrated PBA as a potential therapeutic agent. PBA has been proposed to work with two main action mechanisms: chemical chaperone and histone deacetylase (HDAC) inhibitor. PBA's HDAC inhibitor activity prevents neurons against ER stress and inhibits GSK3β, which protects tau phosphorylation and restores plasticity in the neurons. PBA also upregulates downstream synaptic plasticity markers such as GluR1 subunit AMPA receptor, PSD95, and MAP-2, resulting in a proper hippocampal function and memory impairment reversal(18). Another study suggests that ER stress suppresses γ-secretase mediated APP proteolysis resulting in loss of function affiliated with mutations of the genetically heritable family AD. PBA mitigates ER tension and facilitates protein trafficking via the secretory pathway, together with PBA-mediated stimulation of a/γ-cleavage(19). Moreover, PBA reversed the observed abnormalities in tau and autophagy, behavioral deficits, and loss of synapsin 1 in Tau35 new mouse model of tauopathy(20).

Parkinson's disease. PBA acts as a chemical chaperone preventing misfolded α-synuclein aggregation(17). Preclinical studies showed that it halted disease progression in a chronic PD mouse model and may have therapeutic benefit in PD(16). In mice, PBA treatment led to a significant increase in brain DJ-1 levels and protected dopamine neurons against 1-methyl 4-phenyl 1,2,3,6-tetrahydropyridine (MPTP) toxicity. In a transgenic mouse model of diffuse Lewy body disease, long-term administration of PBA reduced alpha-synuclein aggregation in brain and prevented age-related deterioration in motor and cognitive function(21).

Amyotrophic Lateral Sclerosis. In vivo studies showed that PBA significantly extended survival and improved both the clinical and neuropathological phenotypes in G93A transgenic ALS mice(22). The combined treatment of riluzole and PBA significantly extended survival and improved both the clinical and neuropathological phenotypes in G93A transgenic ALS mice beyond either agent alone(23). A 20-week treatment phase II human trial of PBA found it to be safe and tolerable(24). In the recent double blind, placebo-controlled trial in humans, a combination of PBA with TUDCA resulted in slower functional decline than placebo as measured by the ALSFRS-R score over a period of 24 weeks(25).

Huntington's disease. Preclinical studies of PBA in a transgenic mouse model of HD significantly extended survival and attenuated both gross brain and neuronal atrophy after onset of symptoms(26). The drug appears to be safe and well-tolerated in HD patients up to 15 g/day(27).

A difficulty with the therapeutic use of PBA in humans is the high dosage requirement, up to 15 g/day. In an embodiment, it may be possible to reduce this dosage by the use of an extended release formulation as disclosed by Truog(28). For example, PBA can be formulated with a hydrophilic polymer. The hydrophilic polymer may be at least one cellulose ether polymer selected from the group consisting of methylcellulose, hydroxyethyl cellulose and hydroxypropyl cellulose. The hydrophilic polymer may be selected from the group consisting of non-cellulose polysaccharides, polyethylene oxide/glycol, polyvinyl alcohols and acrylic acid co-polymers. Alternatively, PBA can be conjugated or covalently linked to a polyethylene glycol (PEG) to form a pegylated PBA. In another example, a PBA extended-release formulation can be an osmotic device, which is a tablet having a core of an active ingredient combined with an osmotic agent. An osmotic tablet is coated with a semipermeable membrane that allows water to pass through the membrane into the core but not out of the membrane. The water that enters the tablet elevates the osmotic pressure from the osmotic agent inside the coated tablet. An orifice in the tablet relieves the pressure and allows the active agent to flow out of the tablet at a controlled rate. Other extended or controlled release systems are possible. As used herein, the term “extended release” is synonymous with “controlled release,” “sustained release,” and “modified release.”

A dose of PBA for clinical use is 0.5 to 7.5 g orally twice daily, or 1.0 g to 5.0 g twice daily, or 3.0 g twice daily.

Bile acids also act as chemical chaperones. Bile acids include tauroursodeoxycholic acid (TUDCA), ursodeoxycholic acid (UDCA), and deoxycholic acid (DCA).

Tauroursodeoxycholic acid (TUDCA), an endogenous bile acid, is a strong neuroprotective agent. TUDCA is a taurine conjugate of ursodeoxycholic acid (UDCA). It is permeable to the blood-brain barrier and has a low toxicity profile(29), (30). TUDCA has been shown to have beneficial effects in AD, ALS, PD and HD. These disorders share the pathologies of accumulation of protein aggregates in the brain, neuroinflammation and mitochondrial dysfunction(31).

Bile acids such as UDCA and TUDCA have been shown to suppress the toxic aggregation of misfolded proteins in various animal models of neurodegenerative diseases. These bile acids safeguard neurons also by reducing the synthesis of reactive oxygen species, mitigating mitochondrial damage, and inhibiting apoptosis through both the intrinsic and extrinsic pathways(29). Moreover, TUDCA and UDCA substantially reduced PrP conversion in cell-free aggregation assays, and in chronically and acutely infected cell cultures. TUDCA and UDCA also reduced neuronal loss in prion-infected cerebellar slice cultures suggesting they may have a therapeutic role in prion diseases(32). TUDCA in combination with PBA has also been found to reduce reactive oxygen metabolite-mediated oxidative damage in neurons and improve neuronal viability.(33)

Alzheimer's disease. TUDCA inhibits the accumulation of amyloid β (Aβ) deposits in AD. It also prevents glial activation and a loss of neuronal integrity. Connective tissue growth factor (CTGF) is a cysteine-rich protein that has been shown to promote the activity of γ-secretase and Aβ neuropathology. (34) TUDCA can conflict with the processing of APP and it has an inhibitory effect on the expression of CTGF. TUDCA decreases the production of amyloidogenic APP-CTF-γ and APP-CTF-β, direct precursors of Aβ. TUDCA or similarly acting compounds such as UDCA could be therapeutically useful γ-secretase modulators(34);(35). In APP/PS1 mice, an experimental model of AD, a 6-month treatment with 0.4% of TUDCA in diet prevented Aβ plaque accumulation in the brain(34), (35). An improvement in the spatial, recognition and contextual memory was also observed in APP/PS1 mice after this treatment.

The fundamental basis of TUDCA's neuroprotective ability is more focused on its anti-apoptotic properties than on ER stress relieving activity. TUDCA exerts anti-apoptotic effects by minimizing nuclear fragmentation; by reducing caspase 2 and 6 activations; and by modulating p53, Bcl-2, and Bax activity(36). The treatment with 100 UM of TUDCA for 12 h can significantly decrease Aβ peptide-associated apoptosis in cortical neurons(37).

Therefore, both in vitro and in vivo results show the effectiveness of TUDCA in decreasing apoptosis, attenuating Aβ production and deposition, TAU hyperphosphorylation, and loss of synaptic function.

Parkinson's disease. Duan et al showed that the application of TUDCA facilitates the survival of DA neurons in vitro and in vivo conditions(38). TUDCA-treated group demonstrated increase in the number of tyrosine hydroxylase positive neurons, used as a marker for dopamine, norepinephrine, and epinephrine-containing neurons(39); and a reduction in the number of apoptotic cells.

In MPTP mouse model, pre-treatment with TUDCA (50 mg/kg for 3 days) significantly reduced neurodegeneration of the nigral dopaminergic neurons caused by MPTP, as well as reduced dopaminergic fiber loss and ameliorated motor performance and symptoms typical of PD, such as spontaneous activity, ability to initiate movement and tremors.(40). TUDCA treatment also prevented the production of MPTP-dependent ROS in GSTP null mice(40). TUDCA-dependent mitoprotective effects have also been observed in primary mouse cortical neurons and neuroblastoma cell line SH-SY5Y(41). All this makes TUDCA useful in attenuating mitochondrial dysfunction and ROS production as well as inhibiting multiple proteins involved in apoptosis.

Amyotrophic Lateral Sclerosis. In a small double-blind, placebo-controlled study of TUDCA in riluzole-treated patients, TUDCA was well tolerated. The proportion of responders was higher with TUDCA (87%) than with placebo (P=0.021; 43%). At study end baseline-adjusted ALSFRS-R was significantly higher (P=0.007) in TUDCA than in placebo groups. Comparison of the slopes of regression analysis showed slower progression in the TUDCA than in the placebo group (P<0.01)(42).

Huntington's disease. The treatment with TUDCA exhibited a significant reduction in apoptosis in a 3-NP rat model of HD, as well as preserved striatal mitochondria morphology(43). In cultured striatal cells, TUDCA treatment prevented 3-NP-mediated neuronal death(43). The treatment with 500 mg/kg of TUDCA also generated neuroprotective effects in the R6/2 transgenic mice model of HD(44). TUDCA-treated mice exhibited significant improvement in locomotor and sensorimotor deficits.

A dosage of TUDCA may be to 0.25 g to 2 g per day in two divided doses. A dosage of UDCA may be 5 mg to 15 mg/kg per day administered in two to four divided doses.

### Molecular Chaperones/HSP Co-Inducers

Molecular chaperones are proteins that assist the conformational folding or unfolding of large proteins or macromolecular protein complexes. Heat Shock Proteins (HSP's) function as molecular chaperones and help to maintain protein homeostasis within the cell. HSP's provide protection against protein aggregation, facilitate the folding of newly synthesized polypeptides and refolding of proteins that have been damaged, and target and sequester proteins that have been severely damaged for degradation(45). HSPs are naturally occurring in the human body. HSP co-inducers are drugs that enhance activation of HSP under conditions of stress. Two experimental HSP co-inducers that may be of value in this invention are arimoclomol and bimoclomol.

Alzheimer's disease. Small HSP's have been reported to inhibit Aβ aggregation and effectively block the cerebrovascular toxicity of Aβ(46). Both HSP70 and HSP90 facilitate solubilization of tau proteins and promote partitioning of tau into a productive folding pathway to form microtubules, thereby preventing aggregation of these proteins into NFTs. In addition, HSP70 and HSP90 are associated with accelerated tau degradation(47). Therefore, it is reasonable to expect that induction of HSP by arimoclomol would further reduce aggregation of Aβ and tau proteins.

Parkinson's Disease. Coexpression of HSP70 with alpha-synuclein has been shown to inhibit the formation of alpha-synuclein fibrils and reduce their toxicity, both in vitro as well as in vivo in Drosophila and mouse, possibly by binding of HSP70 to prefibrillar alpha-synuclein(48),(49),(50). Therefore, it is reasonable to think that induction of HSP by arimoclomol would further reduce aggregation of misfolded alpha-synuclein.

Amyotrophic Lateral Sclerosis. In SOD1 preclinical models, treatment with arimoclomol from early (75 days) or late (90 days) symptomatic stages significantly improved muscle function. Treatment from 75 days also significantly increased the lifespan of SOD(G93A) mice(51). In a small randomized clinical trial in patients with rapidly progressive SOD1 ALS, survival favored arimoclomol with a hazard ratio of 0.77 (although not statistically significant). ALSFRS-R and FEV6 declined more slowly in the arimoclomol group as well(52).

Huntington's Disease. Increased levels of HSP40, HSP60, HSP70, and HSP100 have been shown to inhibit polyglutamine-induced huntingtin protein aggregation seen in HD and thus impede disease progression(53),(54),(55). Therefore, it is reasonable to expect that induction of HSP by arimoclomol would further reduce aggregation of huntingtin.

A dose of arimoclomol may be 30 mg to 600 mg per day in three divided doses(52, 56).

### GLP-1 Agonists

Glucagon-like peptide-1 (GLP-1) is a 30- or 31-amino acid peptide hormone deriving from the tissue-specific posttranslational processing of the proglucagon peptide. It is produced and secreted by intestinal enteroendocrine L-cells and certain neurons within the nucleus of the solitary tract in the brainstem upon food consumption. There is a receptor of GLP-1, the glucagon-like peptide-1 receptor (GLP1R). This receptor protein is found on beta cells of the pancreas and on neurons of the brain. It is a member of the glucagon receptor family of G protein-coupled receptors.

GLP-1 agonists act as a survival factor for dopaminergic neurons by functioning as a microglia-deactivating factor(57). GLP-1 agonists can reduce inflammation(58), the accumulation of misfolded neuroproteins, (59) and improve mitochondrial function(60). Recent preclinical studies suggest that GLP-1 agonists such as exenatide may be a valuable therapeutic agent for several neurodegenerative conditions. In a recent clinical trial, exenatide had positive effects on motor scores in Parkinson's disease patients (61)

A leading GLP-1 agonist that may be of value in this invention is exenatide (also called exendin-4), an FDA-approved therapy for diabetes mellitus. Exenatide is a 39-amino acid peptide. Exenatide is normally administered by injection. Experiments have been made on oral delivery technologies for this peptide. (62)

GLP-1 agonists that may be of value in this invention include:
Exenatide (Byetta), taken by injection twice daily
Exenatide extended release (BYDUREON BCISE), taken by injection weekly, BYDUREON BCISE is a suspension of exenatide extended-release microspheres in an oil-based vehicle of medium chai triglycerides.
ORMD-0901—(oral exenatide taken by mouth once daily)
NLY01, a pegylated exenatide analogue.(63) NLY01 has an extended half-life compared to exenatide and penetrates the blood brain barrier.
Dulaglutide (Trulicity), taken by injection weekly
Semaglutide (Ozempic), taken by injection weekly
Semaglutide (Rybelsus), taken by mouth once daily
Liraglutide (Victoza), taken by injection daily
Lixisenatide (Adlyxin), taken by injection daily

A dosage regimen of exenatide may be 5-10 mcg twice daily administered by injection, or 2 mg administered once per week for extended release exenatide.

Alzheimer's disease. Exenatide administration (100 μg/kg twice per day) to FAD transgenic mice prevented cognitive decline after 16 weeks of treatment. Aβ1-42 deposition and synapse damage in the hippocampus was significantly alleviated. Furthermore, exenatide treatment improved mitochondrial morphology, relieved oxidative damage, corrected mitochondrial energy deficit, as well as normalized mitochondrial dynamics. In a small 18-month double-blind randomized placebo-controlled Phase II clinical trial, Exenatide was safe and well-tolerated. It showed reduction of Aβ42 in extracellular vesicles(64).

Parkinson's disease. PBA may act as a survival factor for dopaminergic neurons by functioning as a microglia-deactivating factor(57). It also can reduce inflammation(58), the accumulation of α-synuclein (59) and improve mitochondrial function(60). In a recent clinical trial, Exenatide had positive effects on motor scores in Parkinson's disease patients(61).

Amyotrophic Lateral Sclerosis. Exenatide proved to be neurotrophic and neuroprotective in NSC-19 cells, elevating choline acetyltransferase (ChAT) activity and protecting cells from hydrogen peroxide-induced oxidative stress and staurosporine-induced apoptosis. SOD1 mice treated with exenatide showed improved glucose tolerance and normalization of behavior, as assessed by running wheel, compared to control ALS mice. Furthermore, the drug attenuated neuronal cell death in the lumbar spinal cord. Immunohistochemical analysis demonstrated the rescue of neuronal markers, such as ChAT, associated with motor neurons(65).

Huntington's disease. In transgenic mouse model of HD (N171-82Q), exenatide treatment ameliorated abnormalities in peripheral glucose regulation and suppressed cellular pathology in both brain and pancreas. The treatment also improved motor function and extended the survival time of the Huntington's disease mice. These clinical improvements were correlated with reduced accumulation of mHtt protein aggregates in both islet and brain cells(66). up to 2 mg subcutaneously once a week.

### Iron Chelators

Disruption of brain iron homeostasis leading to abnormal iron accumulation in the brain is implicated in neurodegeneration. Misfolded proteins lead to over-accumulation of intracellular iron(II) in the brain which in turn leads to formation of reactive oxygen species (ROS) and neurodegeneration(11), (12). Also, excess iron is removed from the brain by the natural iron chelator neuromelanin(13),(14). Once neuromelanin is saturated with iron, it causes activation of microglia which leads to formation of reactive oxygen species (ROS) as well as neuroinflammation which in turn results in more brain iron accumulation closing the feedback loop (15) (FIG. 1). Thus, a drug that chelates excess iron in the brain and deactivates or removes iron ions may be of value in interrupting this cycle and treating or preventing neurodegeneration.

Deferiprone (DFP) is an FDA-approved iron chelator therapy for systemic iron overload. DFP has advantage among other iron chelators for its ability to cross membranes, including the blood brain barrier, (67) and to chelate components of the cellular labile iron pool in brain tissue(68).

Other Iron chelators that may be of value in this invention include:
Deferoxamine (DFO), desferoxamine, deferasirox, and clioquinol
8-Hydroxyquinolines analogs such as clioquinol, VK28,
M10 (containing a peptide NAPVSIPQ and an iron-chelating moiety),
Prochelators such as SIH-B and BSIH (derived from salicylaldehyde isocotinoyl hydrazine which is then converted to the active non-specific iron chelator SIH during oxidative stress),
Aroylhydrazones

Alzheimer's Disease. Excess iron upregulates gene expression of amyloid precursor protein, shifts its physiologic non-amyloidogenic processing toward amyloidogenic cleavage that produces Aβ peptides, and contributes to the misfolding of Aβ peptides and production of insoluble Aβ plaques(69). Abnormal iron deposition has been detected in Aβ plaques in histologic evaluation of post-mortem brains from AD patients(70). In mouse model of tauopathy, DFP significantly reduced anxiety-like behavior, and improved cognitive function. This was accompanied by a decrease in brain iron levels and sarkosyl-insoluble tau(71), (72). In a Phase-II trial with Alzheimer's disease patients, treatment with the iron-copper chelator clioquinol resulted in stabilization of Alzheimer's Disease Assessment Scale scores, compared to placebo-treated controls. In addition, plasma Aβ1-42 levels declined in the clioquinol-treated group(73).

Parkinson's Disease. Excess brain iron accumulation contributes to neurodegeneration by inducing the aggregation of alpha-synuclein (74) and formation of Lewy bodies(75). DFP can chelate excessive brain iron from substantia nigra where it is believed to also cause reactive oxygen species (ROS) production and oxidative stress on dopaminergic neurons in Parkinson's Disease patients. In a small double-blind placebo controlled clinical trial in PD patients, DFP showed improvement in both substantia nigra iron deposits (as seen on MRI) and motor scores of disease progression(68). A concomitant clinical benefit was noted at 6 months with a three-point improvement in the unified Parkinson's disease rating scale (UPDRS) for motor skills in the early start group (21.6±8) versus the delayed start group (24±6).

Amyotrophic Lateral Sclerosis. In human studies, dysregulation in iron homeostasis has been reported in patients with ALS, including increased serum ferritin(76). MRI studies also support the finding of increased iron in the motor cortex of patients with ALS(77);(78). In a small double blind, placebo-controlled trial, levels of iron, oxidative stress and the neurofilament light chains in the cerebrospinal fluid were lowered after DFP treatment. A decrease in the ALS Functional Rating Scale score was significantly smaller for the first 3 months on DFP treatment compared with placebo. The reduction in manual muscle testing scores was lower in patients on DFP than on placebo(79).

Huntington's Disease. The abnormal huntingtin protein impairs iron homeostasis in the brain and is suggested to upregulate the expression of iron regulatory protein 1, transferrin, and transferrin receptor, which can result in increased iron accumulation(80). Higher iron content was reported in the basal ganglia by all of the studies in both patients with symptomatic HD and pre-symptomatic carriers of HD mutation. A 10-day oral deferiprone treatment in 9-week R6/2 HD mice showed that deferiprone removed mitochondrial iron, restored mitochondrial potentials, decreased lipid peroxidation, and improved motor endurance(81).

A dosage of DFP for clinical use may be 5 mg/kg/day to 30 mg/kg/day divided in two doses.

c-Abl Tyrosine Kinase Inhibitors

In neurodegenerative disorders, normal autophagic flux is altered, resulting in the accumulation of autophagic vacuoles or autophagosomes.(82) Normal autophagy is a dynamic multi-step process that prevents protein accumulation via sequestration into autophagic vacuoles (autophagosomes). Activation of tyrosine kinase can decrease the activity of parkin, an E3 ligase involved in proteasomal and autophagic degradation via protein ubiquitination and autophagosome maturation. Subsequent fusion of the autophagosomes with lysosomes results in protein degradation. Interruption of this process results in accumulation of protein aggregates and neurodegeneration. Downregulation of parkin thus reduces autophagic clearance, which is implicated in neurodegeneration processes.(83)

Tyrosine kinase inhibition activates parkin-mediated clearance of aggregated proteins and/or activates ubiquitination. Activation of parkin by tyrosine kinase inhibitors upregulates protein levels of beclin, thus facilitating autophagic clearance. For example, nilotinib, bosutinib, or a combination thereof activates parkin-mediated clearance of aggregated proteins and/or activates ubiquitination. Studies suggest that nilotinib crosses the blood brain barrier(84, 85) and promotes parkin activity in the central nervous system. Parkin activity promotes autophagic clearance of amyloid beta and alpha-synuclein and causes protective mechanisms for parkin ubiquitination, for example, sequestration of TDP-43 associated with amyotrophic lateral sclerosis (ALS) and frontotemporal dementia. Furthermore, the tyrosine kinase inhibitors rescue brain cells from apoptotic death in neurodegenerative disease. In the case of ALS, the inhibitors increase ubiquitination of TDP-43 and translocate it from the nucleus, where it interacts deleteriously with mRNA and thousands of genes, to the cytosol where it is sequestered.

A c-Abl tyrosine kinase inhibitor that may be useful in this invention is nilotinib and others listed below. Nilotinib is an FDA-approved drug for treating chronic myeloid leukemia (CML). Nilotinib functions as an inhibitor of cluster-Abelson (c-Abl) tyrosine kinase leading to strong autophagy induction and reduction of neuroinflammation. Other c-Abl tyrosine kinase inhibitors that may be of value in this invention include radotinib, vodobatinib (K0706), bafetinib, imatinib, dasatinib, bosutinib, ponatinib, rebastinib, tozasertib, danusertib and Ikt-148009(83).

Alzheimer's disease. In a preclinical study using transgenic mice, Abl inhibition by nilotinib or bosutinib facilitated amyloid clearance and decreased inflammation (86). In a Tg2576 mouse model of AD, overexpressing a mutated form of the human APP, chronic treatment with Nilotinib reduced c-Abl phosphorylation, improved autophagy, reduced Aβ levels and prevented degeneration as well as functional and morphological alterations in dopaminergic neurons(87). In a recent phase 2, randomized, double-blind, placebo-controlled study, amyloid burden was significantly reduced in the frontal lobe compared to the placebo group. Cerebrospinal fluid Aβ40 was reduced at 6 months and Aβ42 was reduced at 12 months in the nilotinib group compared to the placebo. Hippocampal volume loss was attenuated (−27%) at 12 months and phospho-tau-181 was reduced at 6 months and 12 months in the nilotinib group. Nilotinib was safe and achieved pharmacologically relevant cerebrospinal fluid concentrations(88).

Parkinson's Disease. Activity of c-Abl tyrosine kinase is involved either directly or indirectly in increasing α-synuclein levels, intracellular proteins whose toxic misfolded forms are strongly implicated in the pathogenesis of PD. Administration of low-dose nilotinib penetrates the blood-brain barrier and has been shown to reduce inflammation, inhibits brain c-Abl and enhance autophagic clearance of intraneuronal α-synuclein in A53T transgenic mice and lentiviral gene transfer models of PD(89). c-Abl may also be a therapeutic target to mitigate prion-mediated neurotoxicity. In Phase 2 clinical trials for PD, nilotinib was well tolerated and resulted in favorable changes in exploratory biomarkers of PD pathophysiology(85),(90).

Amyotrophic lateral Sclerosis. An animal model study showed that survival of G93A mice was improved by oral administration of dasatinib, a c-Abl inhibitor, which also decreased c-Abl phosphorylation, inactivated caspase-3, and improved the innervation status of neuromuscular junctions. In addition, c-Abl expression in postmortem spinal cord tissues from sporadic ALS patients was increased by 3-fold compared with non-ALS patients(91). In another preclinical study, ROS production, mediated at least in part through mitochondrial alterations, trigged c-Abl signaling and lead to motoneuron death which was prevented by another c-Abl inhibitor—imatinib(92).

Huntington's disease. Given the relationship between Abl and neurodegeneration, Abl inhibition with nilotinib is thought to decrease the accumulation of alpha-synuclein(89). In a preclinical study, overexpression of alpha-synuclein in mouse models of HD enhances the onset of tremors and weight loss(93).

A dosage of nilotinib for clinical use may be 50 mg to 300 mg daily divided in 2 doses.

### Combinations

The above classes of drugs have been previously studied individually for neurodegenerative disorders and showed efficacy in preclinical models of PD, AD, ALS and HD where each of the classes acted on a same or different part of the neurodegenerative process. Those drugs acting on different processes as shown on FIG. 1 are expected to produce a synergistic effect if combined, whereas the drugs acting on the same targets can produce greater additive effect as a combination eliminating the need for increasing the doses of a single drug to achieve the same effect, which may not be tolerable by patients. This invention, therefore, proposes to use different combinations of the representatives of the above classes of compounds such as: PBA, TUDCA, Exenatide, Nilotinib, Arimoclomol and DFP; which are thought to stop or slow individual parts of the neurodegenerative processes seen in PD, AD, ALS and HD as well as tauopathies such as PSP, FTD and CBD. We expect some combinations between two and five drugs shown in Tables 1a and 1b to produce synergistic effects in each of the above ND's. Such effects are expected to be superior to the effects from each individual drug alone. These combinations should result in clinical efficacy in human trials for the above diseases and allow agents to be utilized at lower doses to minimize potential side effects.

TABLE 1a
Proposed combinations of classes of drugs

| # | Combination |
|---|---|
| 1 | Chemical chaperone (PBA), GLP-1 |
| | agonist |
| 2 | Chemical chaperone (PBA), Iron |
| | Chelator |
| 3 | Chemical chaperones (PBA + Bile |
| | Acid), GLP-1 agonist |
| 4 | Chemical chaperones (PBA + Bile |
| | Acid), GLP-1 agonist, Iron chelator |
| 5 | Bile acid, GLP-1 agonist |
| 6 | Bile acid, Iron Chelator |
| 7 | Bile acid, GLP-1 agonist, Iron chelator |
| 8 | GLP-1 agonist, Iron chelator |
| 9 | Chemical chaperone (PBA), GLP-1 |
| | agonist, Iron chelator |
| 10 | Chemical chaperones, (PBA + Bile |
| | Acid), Iron Chelator |
| 11 | Chemical chaperones (PBA + Bile |
| | Acid) |
| 12 | Chemical chaperones, (PBA + Bile |
| | Acid), HSP co-inducer |
| 13 | Chemical chaperone (PBA), HSP |
| | co-inducer |
| 14 | Bile acid, HSP co-inducer |
| 15 | Chemical chaperones (PBA + Bile |
| | Acid), GLP-1 agonist, HSP co- |
| | inducer |
| 16 | Chemical chaperone (PBA), GLP-1 |
| | agonist, HSP co-inducer |
| 17 | Chemical chaperone (PBA), c-Abl |
| | inhibitor |
| 18 | GLP-1 agonist, c-Abl inhibitor |
| 19 | Chemical chaperones (PBA + Bile |
| | Acid), Iron Chelator, c-Abl inhibitor |
| 20 | Chemical chaperones (PBA + Bile |
| | Acid), c-Abl inhibitor |
| 21 | Bile acid, c-Abl inhibitor |
| 22 | Chemical chaperones (PBA + Bile |
| | Acid), GLP-1 agonist, Iron Chelator, c- |
| | Abl inhibitor |
| 23 | Bile acid, GLP-1 agonist, c-Abl inhibitor |
| 24 | Chemical chaperones (PBA + Bile |
| | Acid), GLP-1 agonist, c-Abl inhibitor |
| 25 | Chemical chaperone (PBA), GLP-1 |
| | agonist, c-Abl inhibitor, HSP co- |
| | inducer |
| 26 | Chemical chaperones (PBA + Bile |
| | Acid), c-Abl inhibitor, HSP co-inducer |
| 27 | GLP-1 agonist, c-Abl inhibitor, HSP co- |
| | inducer |
| 28 | c-Abl inhibitor, HSP co-inducer |
| 29 | Bile acid, GLP-1 agonist, c-Abl |
| | inhibitor, HSP co-inducer |
| 30 | Bile acid, GLP-1 agonist, HSP co- |
| | inducer |
| 31 | Chemical chaperone (PBA), GLP-1 |
| | agonist, Iron Chelator, HSP co-inducer |
| 32 | GLP-1 agonist, Iron Chelator, HSP co- |
| | inducer |
| 33 | GLP-1 agonist, HSP co-inducer |
| 34 | Bile acid, GLP-1 agonist, Iron Chelator, |
| | HSP co-inducer |
| 35 | Chemical chaperone (PBA), GLP-1 |
| | agonist, c-Abl inhibitor |
| 36 | Chemical chaperone (PBA), Iron |
| | Chelator, c-Abl inhibitor |
| 37 | Bile acid, GLP-1 agonist, Iron Chelator, |
| | c-Abl inhibitor |
| 38 | Bile acid, Iron Chelator, c-Abl inhibitor |
| 39 | GLP-1 agonist, Iron chelator, c-Abl |
| | inhibitor |
| 40 | Chemical chaperone (PBA), GLP-1 |
| | agonist, Iron chelator, c-Abl inhibitor |
| 41 | Iron chelator, c-Abl inhibitor |
| 42 | Iron chelator, HSP co-inducer |

TABLE 1b
Drug examples from the above classes
(not in the same order as Table 1a).

| # | Combination |
|---|---|
| 1 | PBA, EXD |
| 2 | PBA, DFP |
| 3 | PBA, TUDCA, EXD |
| 4 | PBA, TUDCA, EXD, DFP |
| 5 | TUDCA, EXD |
| 6 | PBA, TUDCA |
| 7 | PBA, EXD, NTB |
| 8 | PBA, DFP, NTB |
| 9 | PBA, TUDCA, EXD, NTB |
| 10 | PBA, TUDCA, EXD, DFP, NTB |
| 11 | TUDCA, EXD, NTB |
| 12 | PBA, TUDCA, NTB |
| 13 | PBA, NTB |
| 14 | TUDCA, NTB |
| 15 | TUDCA, DFP |
| 16 | TUDCA, EXD, DFP |
| 17 | EXD, DFP |
| 18 | PBA, EXD, DFP |
| 19 | PBA, TUDCA, DFP |
| 20 | TUDCA, DFP, NTB |
| 21 | TUDCA, EXD, DFP, NTB |
| 22 | EXD, DFP, NTB |
| 23 | PBA, EXD, DFP, NTB |
| 24 | PBA, TUDCA, DFP, NTB |
| 25 | EXD, NTB |
| 26 | DFP, NTB |
| 27 | PBA, EXT, NTB, ARM |
| 28 | PBA, TUDCA, NTB, ARM |
| 29 | EXD, NTB, ARM |
| 30 | NTB, ARM |
| 31 | TUDCA, EXD, NTB, ARM |
| 32 | TUDCA, EXD, ARM |
| 33 | PBA, EXD, DFP, ARM |
| 34 | EXD, DFP, ARM |
| 35 | EXD, ARM |
| 36 | TUDCA, EXD, DFP, ARM |
| 37 | PBA, TUDCA, ARM |
| 38 | PBA, ARM |
| 39 | TUDCA, ARM |
| 40 | PBA, TUDCA, EXD, ARM |
| 41 | PBA, EXD, ARM |
| 42 | DFP, ARM |

Codes:
1. PBA—Sodium Phenylbutyrate
2. TUDCA—Tauroursodeoxycholic acid
3. EXD—Exendin-4 (Exenatide)
4. DFP—Deferiprone
5. NTB—Nilotinib
6. ARM—Arimoclomol

Some specific embodiments of combinations expected to be of utility in this invention include combinations of the following drugs: sodium phenylbutyrate (PBA) and exenatide (EXD); PBA, tauroursodeoxycholic acid (TUDCA), and EXD; nilotinib (NL) and TUDCA; EXD and TUDCA; EXD, NL, and TUDCA; PBA, EXD, and deferiprone (DFP); PBA, NL, and TUDCA; EXD and NL; PBA, EXD, and NL; PBA, EXD, TUDCA, and DFP; PBA, arimoclomol (ARM) and TUDCA; and EXD and ARM.

### Examples

### Neurite Growth and Branching Study in PD Model

Given the conceptual similarities in pathological pathways occurring in the neurodegenerative diseases described above and the provided evidence from the literature showing that the six medications individually from the five different classes exhibit neuroprotective effect in different preclinical models of PD, AD, ALS and HD we believe that various combinations of those six medications should also show superior efficacy in all the above diseases compared to each individual drug alone. To prove this, we chose an in-vitro model of Parkinson's Disease as an example to illustrate advantage of combining these drugs to achieve better efficacy, testing for neurite growth and neurite branching as described below.

### In-Vitro Study Protocol

In order to evaluate some of the above-proposed combinations of compounds from Table 1b, we conducted an in vitro study to evaluate the neuroprotective effects of 10 conditions of treatment (Tables 3) against MPP+-induced neurodegeneration toxicity on human dopaminergic neurons (iCell®) DopaNeurons: iPS cell-derived human midbrain floorplate dopaminergic neurons). Additional combinations listed in Tables 1 a and b will be studied in the near future.

MPP+(1-methyl-4-phenylpyridinium) is a known neurotoxin which acts by interfering with oxidative phosphorylation in mitochondria by inhibiting complex I, a protein in the membrane of mitochondria in dopaminergic neurons in the substantia nigra.(94) The inhibition of mitochondrial function leads to the depletion of ATP and eventual cell death. MPP+ ultimately causes Parkinsonism in primates by killing certain dopamine-producing neurons in the substantia nigra. The ability of MPP+ to induce Parkinson's disease has made it an important compound in Parkinson's research.

iCell DOPA neurons are neural floor plate-derived midbrain dopaminergic neurons generated from human induced pluripotent stem cells (iPSCs). Dopaminergic neurons, specifically those located in the floor plate-derived midbrain are implicated in neurological disorders such as Parkinson's disease, MSA and DLBD among others. Thus iCell DopaNeurons provide a highly relevant in vitro model to investigate these types of pathologies. The iCellDopa neurons were supplied from from FUJIFILM Cellular Dynamics.

Each compound in Table 2 was studied in the vehicle solution indicated in the last column.

Three different measurements were made in the experiments. Neurite length in mm was measured, which is a measure of increase in dendril length of neurons. Healthy cells exhibited significant increases in neurite length under the control conditions. Secondly, neurite branching was measured. Healthy cells exhibited significant increases in neurite branching under the control conditions. Thirdly, cytolysis of the cells was measured. Healthy cells exhibited reduced cytolysis. Experimental conditions were measured relative to the results of healthy cells in the studies.

TABLE 2
Compounds Studied do date

| Com- | | | | Testing | |
|---|---|---|---|---|---|
| pound | | | | concen- | Final |
| No. | Compound | Provider | Stock | tration | Vehicle |
| 1 | Sodium | TOCRIS | 100 mM in | 500 μM | 0.5% |
| | Phenyl- | cat#2682 | H2O | (literature | H2O |
| | butyrate | | (TOCRIS | provided) | |
| | | | datasheet) | | |
| 2 | TUDCA | Cat# | 250 mM | 50 μM | 0.02% |
| | | 580549 | | | H2O |
| 3 | Exendin-4 | SIGMA | 200 μM in | 100 nM | Culture |
| | (Exenatide) | cat#E7144 | culture | (PORSOLT | medium |
| | | | medium | insulin | |
| | | | (PORSOLT | assay) | |
| | | | experience) | | |
| 4 | Deferipone | Sigma | 50 mM | 50 μM | 0.1% |
| | | Aldich | DMSO | | DMSO |
| | | Cat# | | | |
| | | Y0001976 | | | |
| MPP+ | MPP+ | Sigma | | 100 μM | Culture |
| | | D048 | | | medium |

Control and Vehicle. The culture medium was BrainPhys™ Neuronal Medium, supplied by Stemcell Technologies, www.stemcell.com. Vehicles were culture medium alone, 0.5% H₂O and 0.02% H₂O and 0.16% DMSO in culture medium.

Dosage forms preparation. The solubility of the test substances was checked before each experiment. Each test substance was prepared at the stock solution and working concentration described below to evaluate its solubility. The test substances were prepared freshly on the day of the experiment.

Sodium Phenylbutyrate was prepared at a stock solution of 100 mM in 100% H₂O (stock solution at 200×).

Exendin-4 (Exenatide) was prepared at a stock solution of 200 μM in culture medium (stock solution at 2 000 000×).

Deferipone was prepared at a stock solution of 50 mM in 100% DMSO (stock solution at 1000×).

Tauroursodeoxycholic acid was prepared at a stock solution of 250 mM in 100% H₂O (stock solution at 5000×).

Procedure. iCell DOPA neurons were thawed and cultured following the provider's instructions in Brainphys medium+provided supplements+1% N2 supplement (Stemcell Technologies)+penicillin/streptomycin+Laminin. They were plated at 20000 cells per well of a 384 well plate (pre-coated with poly-D-lysine and Laminin) in 70 μL of growth medium. Cells were incubated at 37° ° C./5% CO2 in a humidified cell culture incubator. Half of the culture medium was changed twice a week.

MPP+-induced neurotoxicity: 24 hours after neuronal plating, half of the medium was removed and the test compounds were applied with MPP+ treatment, both concentrated at 4×, were added to the wells. The various combinations and conditions studied are tabulated in Table 3. For each well, five aliquots were added, corresponding columns A-E in Table 3. In each row, either the compound indicated dissolved in its vehicle noted in Table 2 was added, or only a vehicle was added as indicated. Column E is an indicator of whether MPP+ was added. If not, only the medium was added.

For the evaluation of conditions 8, 10,12, 22, 24, Neurite outgrowth was followed for subsequent 48 hours using an Incucyte Zoom platform with one phase contrast image every 4 hours, using a 20× objective. After 48 hours, the medium containing test compounds and MPP+ was removed and replaced by fresh medium containing a fluorescent cytolysis marker (red fluorescence) and a live cell marker (green fluorescence). For the evaluation of conditions 10, 12, 24, 38, 40, 51-53, Neurite outgrowth was followed similarly for subsequent 72 hours after MPP+ was added.

TABLE 3
Experimental Combinations studied

| Experiment No. | A | B | C | D | E |
|---|---|---|---|---|---|
| Control for | 0.5% | 0.02% | Medium | 0.2% | Medium |
| #8, 10, 12 | H2O | H2O | | EtOH | |
| (Control/NT) | | | | | |
| Control for | 0.5% | 0.02% | Medium | 0.2% | MPP+ |
| #8, 10, 12 | H2O | H2O | | EtOH | |
| (Control/MPP+) | | | | | |
| #8 | 1 | 0.02% | 3 | 0.2% | MPP+ |
| | | H2O | | EtOH | |
| #10 | 1 | 0.02% | Medium | 0.2% | MPP+ |
| | | H2O | | EtOH | |
| #12 | 0.5% | 0.02% | 3 | 0.2% | MPP+ |
| | H2O | H2O | | EtOH | |
| Control for | 0.5% | 0.02% | Medium | 0.1% | Medium |
| #22 to #53 | H2O | H2O | | DMSO | |
| Control for | 0.5% | 0.02% | Medium | 0.1% | MPP+ |
| #22 to #53 | H2O | H2O | | DMSO | |
| #22 | 1 | 0.02% | Medium | 4 | MPP+ |
| | | H2O | | | |
| #24 | 0.5% | 0.02% | Medium | 4 | MPP+ |
| | H2O | H2O | | | |
| #38 | 0.5% | 0.02% | 3 | 4 | MPP+ |
| | H2O | H2O | | | |
| #40 | 1 | 0.02% | 3 | 4 | MPP+ |
| | | H2O | | | |
| #51 | 0.5% | 2 | Medium | 0.2% | MPP+ |
| | H2O | | | EtOH | |
| #52 | 1 | 2 | Medium | 0.2% | MPP+ |
| | | | | EtOH | |
| #53 | 1 | 2 | 3 | 0.2% | MPP+ |
| | | | | EtOH | |
| Positive control | PCI | — | — | — | MPP+ |
| | (30 μM) | | | | |

Table 3 compound key (see also Table 1):
1. Sodium Phenylbutyrate (500 μM)
2. TUDCA (50 μM)
3. Exendin-4 (Exenatide) (100 nM)
4. Deferiprone (50 μM)

The conditions were performed in 8 wells of a 384 well plate, that is, each test condition studied in Table 3 was repeated eight times. Control/NT are the cells in the vehicle (as described above), with and without MPP+. This phase of the study tested conditions 8, 10, 12, 22, 24, 38, 40 and 51-53 in two different plates (as described below). Other combinations proposed in Table 1b will be studied in a later round of experiments.

TABLE 4
Drug Combinations studied

| Combination number | Drugs in combination |
|---|---|
| 8 | PBA, EXD, MPP+ |
| 10 | PBA, MPP+ |
| 12 | EXD, MPP+ |
| 22 | PBA, DFP, MPP+ |
| 24 | DFP, MPP+ |
| 38 | EXD, DFP, MPP+ |
| 40 | PBA, EXD, DFP, MPP+ |
| 51 | TUDCA, MPP+ |
| 52 | PBA, TUDCA, MPP+ |
| 53 | PBA, TUDCA, EXD, MPP+ |

Legend:
PBA—Sodium Phenylbutyrate
EXD—Exendin-4 (Exenatide)
DFP—Deferiprone
NTB—Nilotinib (not studied in this table)
TUDCA—Tauroursodeoxycholic acid
MPP+—1-methyl-4-phenylpyridinium
PCI—Pan Caspase Inhibitor (Positive Control)

Assay endpoints and data analysis. Phase contrast images were analyzed at each time point to determine the neurite length and number of branch points per mm². Kinetic data was plotted and kinetics were normalized by subtracting the value of the first data point (at time of treatment), allowing to measure changes in neurite outgrowth only from the onset of the treatment, starting at zero. Area Under Curve (AUC) of kinetic data was obtained and used for plotting compound's effects and perform statistical analyzes.

Data was normalized to control conditions as there is basal cytolysis in the cell culture after thawing.

Fluorescently immuno-stained cells were imaged on a high content imaging platform. Individual segmentation of cells was performed by image analysis of the MAP2 and NeuN staining. The number of neurons and the neurite length were measured.

Statistical analysis. We used one-way ANOVA followed by Bonferroni's multiple comparisons test.

Results for MPP+-Induced Neurotoxicity on iCellDopa Neurons

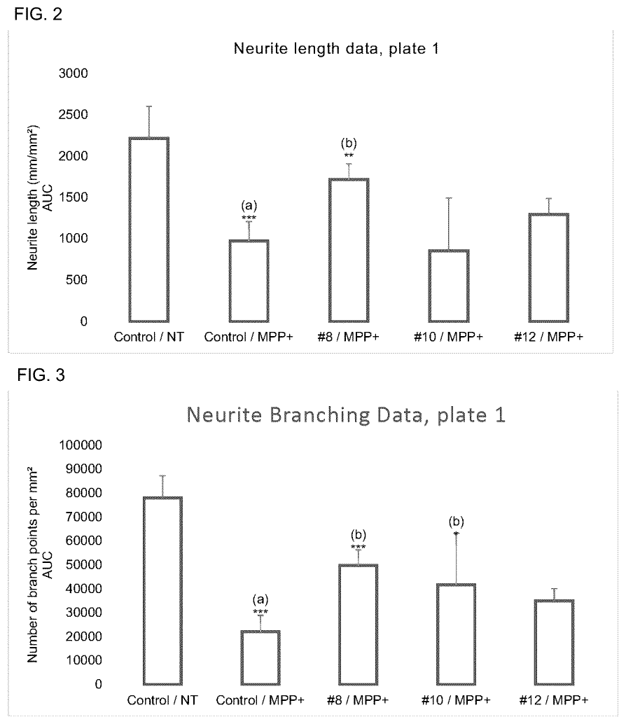

FIGS. 2 and 3 are bar graphs of neurite length and neurite branching for conditions 8, 10 and 12 (Plate 1). Raw data is in Tables 5.1 and 5.2. MPP+ at 100 M (the bar Control/MPP+) induced a high inhibition of neurite outgrowth (AUC of the neurite length and the number of branch), as compared with Control/non-treated (NT) conditions (p≤0.001 for both conditions).

In condition 8 (phenylbutyrate and exenatide), the neurite length and the number of branch points were 75% increased, as compared with MPP+ alone (p≤0.05 and p≤0.001, respectively). However, the drugs tested individually (conditions 10 (PBA alone) and 12 (exenatide alone)) showed much less protective effect than combination 8.

TABLE 5.1
Neurite length data, plate 1 (bar graph in FIG. 2)
Neurite length (mm/mm²) - Baseline-corrected
AUC (0-72 h)

| | | | | | Stat vs | Stat vs |
|---|---|---|---|---|---|---|
| | | | | Stat vs | Control/ | Control/ |
| Treatment | Mean | ± | S.D. | NT/NT | NT | MPP+ |
| Control/NT | 2215.6 | ± | 387.4 | NS | | |
| Control/MPP+ | 977.2 | ± | 230.6 | | *** | |
| #8/MPP+ | 1718.7 | ± | 187.9 | | | ** |
| #10/MPP+ | 853.7 | ± | 643.8 | | | NS |
| #12/MPP+ | 1294.5 | ± | 196.2 | | | NS |

NS = Not Significant; * = p < 0.05; ** = p < 0.01; *** = p < 0.001.
One-way ANOVA followed by Bonferroni's multiple comparisons test in case of significant effect.

TABLE 5.2
Neurite branch points data, plate 1 (bar graph in FIG. 3)
Number of branch points per mm²—Baseline-corrected
AUC (0-72 h)

| | | | | Stat vs | Stat vs |
|---|---|---|---|---|---|
| | | | Stat vs | Control/ | Control/ |
| Treatment | Mean | ±S.D. | NT/NT | NT | MPP+ |
| Control/NT | 78045 | ±9151.5 | NS | | |
| Control/MPP+ | 22082 | ±6853.7 | | *** | |
| #8/MPP+ | 49743.7 | ±6618.8 | | | *** |
| #10/MPP+ | 41712.8 | ±21469.3 | | | * |
| #12/MPP+ | 34983.1 | ±5097.8 | | | NS |

NS = Not Significant;
* = p < 0.05;
** = p < 0.01;
*** = p < 0.001.
One-way ANOVA followed by Bonferroni's multiple comparisons test in case of significant effect.

Based on these results for neurite length (Table 5.1 and FIG. 2), condition 8 is 75% better than control/MPP+ and exenatide alone (condition 12) is 32% better than control (FIG. 2). For the number of branch points tabulated in Table 5.2, condition 8 was 125% better than control/MPP+. Phenylbutyrate alone (#10) is 89% better than control and exenatide alone (condition 12) is 58% better than control.

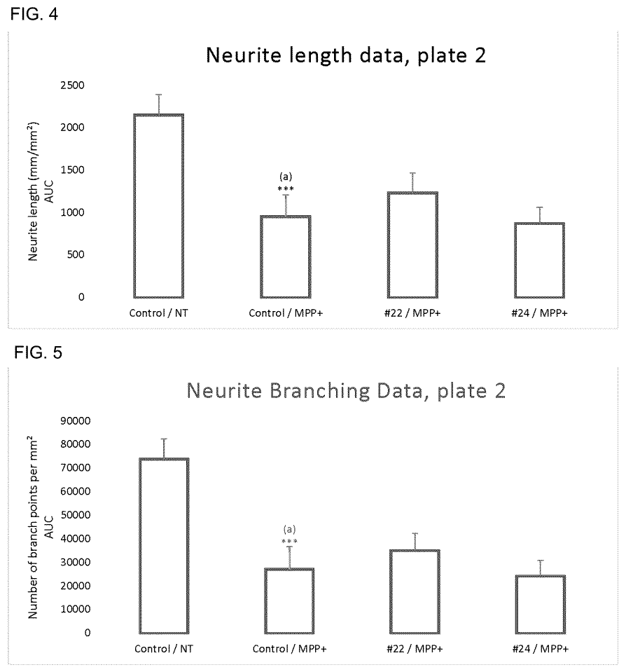

FIGS. 4 and 5 are bar graphs of neurite length and neurite branching for conditions 22 and 24 (Plate 2). Raw data is in Tables 6.1 and 6.2. MPP+ at 100 μM induced a high inhibition of neurite outgrowth (AUC of the neurite length and the number of branch), as compared with Control/NT conditions (p≤0.001 for both conditions).

Condition 22 (the combination of PBA and deferiprone) showed a slight but not statistically significant increase in neurite length (FIG. 4) and neurite branching (FIG. 5) compared to the control/MPP+ bar. Condition 22 also showed an increase (not statistically significant) compared to deferiprone alone in condition 24.

TABLE 6.1
Neurite length data conditions 22 and 24 (plate 2) (bar graph in FIG. 4).
Neurite length (mm/mm²) - Baseline-corrected
AUC (0-72 h)

| | | | | | Stat vs | Stat vs |
|---|---|---|---|---|---|---|
| | | | | Stat vs | Control/ | Control/ |
| Treatment | Mean | ± | S.D. | NT/NT | NT | MPP+ |
| Control/NT | 2148.9 | ± | 242.4 | NS | | |
| Control/MPP+ | 955.3 | ± | 254.5 | | *** | |
| #22/MPP+ | 1234.9 | ± | 233.5 | | | NS |
| #24/MPP+ | 874.5 | ± | 190.4 | | | NS |

NS = Not Significant; * = p < 0.05; ** = p < 0.01; *** = p < 0.001.
One-way ANOVA followed by Bonferroni's multiple comparisons test in case of significant effect.

TABLE 6.2
Neurite Branching Data, plate 2 (bar graph in Fig. 5)
Number of branch points per mm²—Baseline-corrected
AUC (0-72 h)

| | | | | Stat vs | Stat vs |
|---|---|---|---|---|---|
| | | | Stat vs | Control/ | Control/ |
| Treatment | Mean | ±S.D. | NT/NT | NT | MPP+ |
| Control/NT | 73864.0 | ±8671.5 | NS | | |
| Control/MPP+ | 27222.8 | ±9644.1 | | *** | |
| #22/MPP+ | 35112.5 | ±7198.1 | | | NS |
| #24/MPP+ | 24282.5 | ±6612.9 | | | NS |

NS = Not Significant;
* = p < 0.05;
** = p < 0.01;
*** = p < 0.001.
One-way ANOVA followed by Bonferroni's multiple comparisons test in case of significant effect.

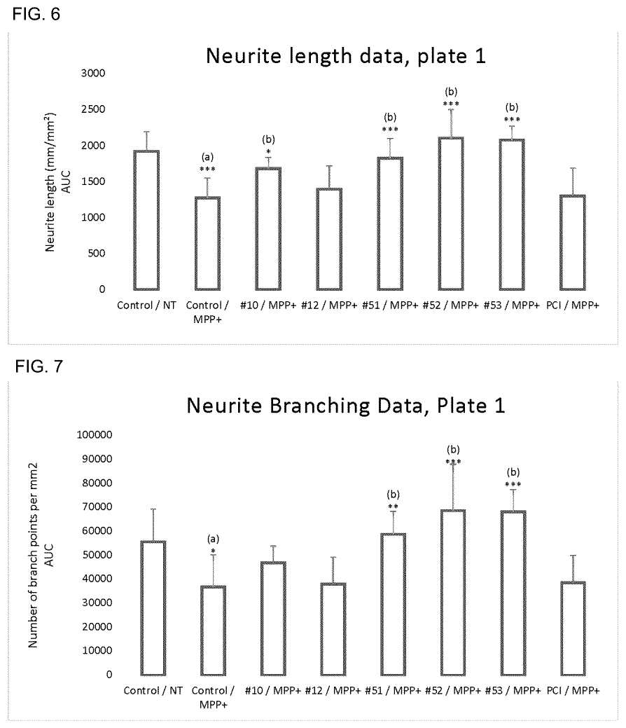

FIGS. 6 and 7 are bar graphs of neurite length and neurite branching for conditions 10, 12, 51, 52, and 53 (Plate 2). Raw data is in Tables 7.1 and 7.2. MPP+ at 100 UM induced a high inhibition of neurite outgrowth (AUC of the neurite length and the number of branch), as compared with Control/NT conditions (p≤0.001 for both conditions). Also plotted is PCI with MPP+. PCI is Pan Caspase Inhibitor Q-VD-OPh,(95) which is a positive control, expected to reduce apoptosis of cells caused by caspases (family of cytosolic aspartate-specific cysteine proteases involved in the initiation and execution of apoptosis) due to exposure to a toxin.

TABLE 7.1
Neurite length data, plate 1 (FIG. 6)

| | | | | Stat vs | Stat vs |
|---|---|---|---|---|---|
| | | | Stat vs | Control/ | Control/ |
| Treatment | Mean | ±S.D. | NT/NT | NT | MPP+ |
| Control/NT | 1920.4 | ±275.8 | NS | | |
| Control/MPP+ | 1272.9 | ±280.0 | | *** | |
| #10/MPP+ | 1681.1 | ±156.1 | | | * |
| #12/MPP+ | 1395.3 | ±326.1 | | | NS |
| #51/MPP+ | 1825.9 | ±273.8 | | | *** |
| #52/MPP+ | 2103.1 | ±399.7 | | | *** |
| #53/MPP+ | 2077.3 | ±195.9 | | | *** |
| PCI/MPP+ | 1301.0 | ±387.8 | | | NS |

NS = Not Significant;
* = p < 0.05;
** = p < 0.01;
*** = p < 0.001.
One-way ANOVA followed by Bonferroni's multiple comparisons test in case of significant effect.

TABLE 7.2
Neurite Branching Data, Plate 1 (FIG. 7)

| | | | Stat vs | Stat vs |
|---|---|---|---|---|
| | | Stat vs | Control/ | Control/ |
| Treatment | Mean ± S.D. | NT/NT | NT | MPP+ |
| Control/NT | 55661.3 ± 13676.2 | NS | | |
| Control/ | 36771.0 ± 13342.2 | | * | |
| MPP+ | | | | |
| #10/MPP+ | 46742.8 ± 7107.5 | | | NS |
| #12/MPP+ | 37977.9 ± 11294.6 | | | NS |
| #51/MPP+ | 58836.3 ± 9596.4 | | | ** |
| #52/MPP+ | 68626.9 ± 19348.6 | | | *** |
| #53/MPP+ | 68062.9 ± 9445.6 | | | *** |
| PCI/MPP+ | 38456.3 ± 11570.7 | | | NS |

NS = Not Significant; * = p < 0.05; ** = p < 0.01; *** = p < 0.001.
One-way ANOVA followed by Bonferroni's multiple comparisons test in case of significant effect.

FIGS. 6 and 7 show promising results for condition 53, a combination of sodium phenylbutyrate, TUDCA, and exenatide. In both neurite length and neurite branching, condition 53 was substantially superior to sodium phenylbutyrate alone (condition 10) and exenatide alone (condition 12). Condition 53 was also superior to TUDCA alone 51 and was comparable to condition 52, a combination of sodium phenylbutyrate and TUDCA. Condition 52 and 53 increased the neurite length (65.2% and 63.2%) and the number of branches (86.6% and 85.1% respectively) compared to MPP+-treated control.

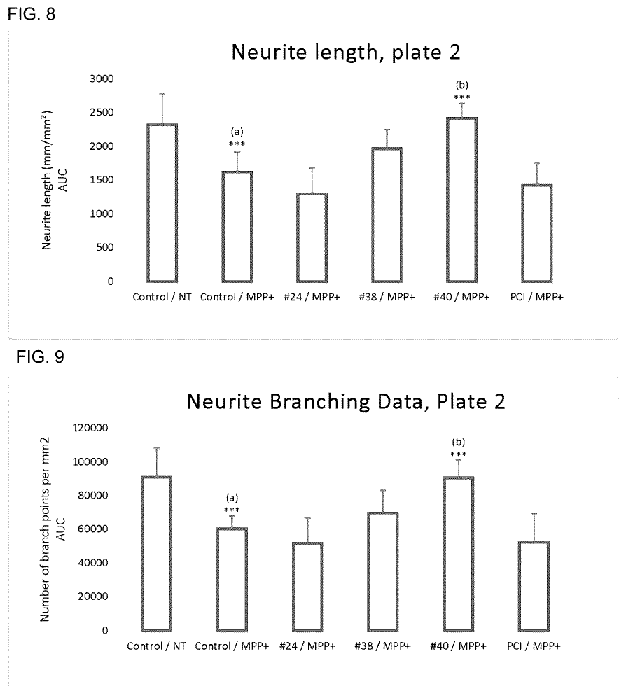

FIGS. 8 and 9 are bar graphs of neurite length and neurite branching for conditions 24, 38, and 40 (Plate 2). Raw data is in Tables 8.1 and 8.2. MPP+ at 100 μM induced a high inhibition of neurite outgrowth (AUC of the neurite length and the number of branch), as compared with Control/NT conditions (p≤0.001 for both conditions). Also shown are results for PCI with MPP+.

TABLE 8.1
Neurite length, plate 2 (FIG. 8)

| | | | Stat vs | Stat vs |
|---|---|---|---|---|
| | | Stat vs | Control/ | Control/ |
| Treatment | Mean ± S.D. | NT/NT | NT | MPP+ |
| Control/NT | 2323.6 ± 458.5 | NS | | |
| Control/ | 1623.9 ± 301.2 | | *** | |
| MPP+ | | | | |
| #24/MPP+ | 1303.0 ± 378.7 | | | NS |
| #38/MPP+ | 1968.3 ± 289.0 | | | NS |
| #40/MPP+ | 2418.3 ± 222.1 | | | *** |
| PCI/MPP+ | 1430.1 ± 321.7 | | | NS |

NS = Not Significant; * = p < 0.05; ** = p < 0.01; *** = p < 0.001.
One-way ANOVA followed by Bonferroni's multiple comparisons test in case of significant effect.

TABLE 8.2
Neurite Branching Data, Plate 2. (FIG. 9)

| | | | Stat vs | Stat vs |
|---|---|---|---|---|
| | | Stat vs | Control/ | Control/ |
| Treatment | Mean ± S.D. | NT/NT | NT | MPP+ |
| Control/NT | 91022.9 ± 17191.7 | NS | | |
| Control/ | 60484.3 ± 7623.4 | | *** | |
| MPP+ | | | | |
| #24/MPP+ | 51711.3 ± 15178.4 | | | NS |
| #38/MPP+ | 69718.3 ± 13499.6 | | | NS |
| #40/MPP+ | 90598.0 ± 10606.3 | | | *** |
| PCI/MPP+ | 52610.1 ± 16660.1 | | | NS |

NS = Not Significant; * = p < 0.05; ** = p < 0.01; *** = p < 0.001.
One-way ANOVA followed by Bonferroni's multiple comparisons test in case of significant effect.

This data shows promising results for combination 40, of sodium phenylbutyrate, exenatide and deferiprone, which shows a synergistic protective effect. The neurite length and the number of branches were increased by 48.9% and 49.7% respectively, as compared with MPP+ alone (p≤0.001 for both). Also shown in FIGS. 8 and 9 are condition 24 (deferiprone alone) and condition 38 exenatide and deferiprone, which were substantially inferior to condition 40.

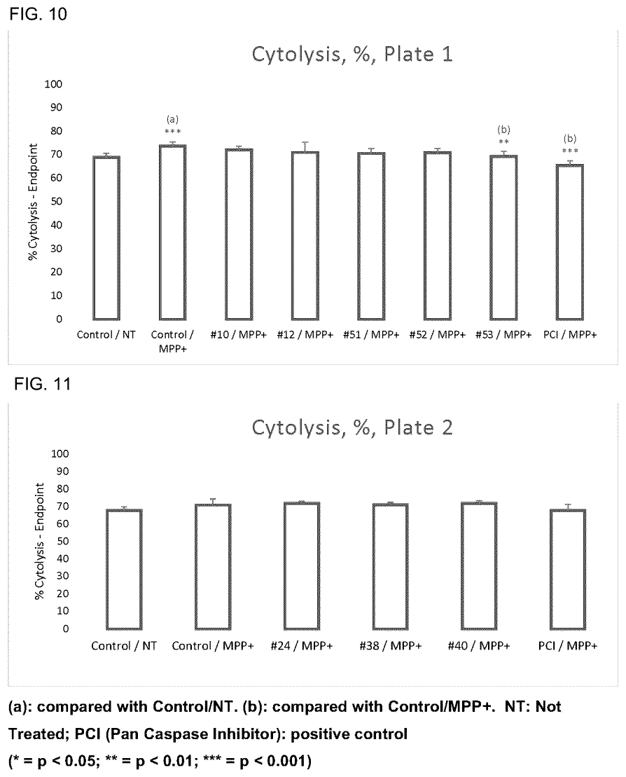

FIG. 10 and Table 9.1 show cytolysis data for conditions 10, 12, 51, 52, 53, and controls including PCI. FIG. 11 and Table 9.2 show cytolysis data for conditions 24, 38, and 40, and controls including PCI.

TABLE 9.1
Cytolysis, %, Plate 1. (FIG. 10)

| | | | Stat vs | Stat vs |
|---|---|---|---|---|
| | | Stat vs | Control/ | Control/ |
| Treatment | Mean ± S.D. | NT/NT | NT | MPP+ |
| Control/NT | 68.82 ± 1.95 | NS | | |
| Control/MPP+ | 73.74 ± 1.85 | | *** | |
| #10/MPP+ | 72.06 ± 1.77 | | | NS |
| #12/MPP+ | 70.99 ± 4.42 | | | NS |
| #51/MPP+ | 70.69 ± 2.02 | | | NS |
| #52/MPP+ | 70.89 ± 1.94 | | | NS |
| #53/MPP+ | 69.31 ± 2.15 | | | ** |
| PCI/MPP+ | 65.56 ± 1.89 | | | *** |

NS = Not Significant; * = p < 0.05; ** = p < 0.01; *** = p < 0.001.
One-way ANOVA followed by Bonferroni's multiple comparisons test in case of significant effect.

TABLE 9.2
Cytolysis, %, Plate 2. (FIG. 11)

| | | | Stat vs | Stat vs |
|---|---|---|---|---|
| | | Stat vs | Control/ | Control/ |
| Treatment | Mean ± S.D. | NT/NT | NT | MPP+ |
| Control/NT | 67.78 ± 2.31 | NS | | |
| Control/MPP+ | 71.00 ± 3.43 | | NS | |
| #24/MPP+ | 71.91 ± 1.28 | | | NS |
| #38/MPP+ | 71.18 ± 1.37 | | | NS |
| #40/MPP+ | 71.81 ± 1.68 | | | NS |
| PCI/MPP+ | 67.93 ± 3.52 | | | NS |

NS = Not Significant; * = p < 0.05; ** = p < 0.01; *** = p < 0.001.
One-way ANOVA followed by Bonferroni's multiple comparisons test in case of significant effect.

Significantly, condition 53 shows a statistically significant reduction of cytolysis (cell death). Conditions 51 and 52 did not reduce cytolysis with statistical significance. While TUDCA alone (condition 51) improved neurite length and branching points as well (43% and 60% respectively), the effect was at least 25% greater when the drugs were combined in condition 53. Reduction of cytolysis in condition 40 was not statistically significant (Table 9.2, FIG. 11).

### α-Synuclein Triplication Cell Line

This experiment measured neurofilament heavy chain signal intensity, a biomarker for neurodegeneration(96),(97) using neurons having triplication of the α-synuclein locus, after treatment of test cells with various combinations of TUDCA, PBA, and EXD.

Human induced pluripotent stem cells (iPSC)-derived dopaminergic (DA) neurons harboring triplication of the α-synuclein locus and an isogenic corrected line were seeded at a density of ˜1,666 cells/mm² on a 96-well glass bottom plate and cultured in SM1-supplemented neurobasal media (STEMCELL Modified-1) for two months before beginning treatment with one or more test drugs, sodium phenylbutyrate (PBA) (500 μM), tauroursodeoxycholic acid (TUDCA) (50 μM), and exendin-4 (EXD) (100 nM) for three months with twice-weekly media changes. We used 12 replicas for each experimental condition and 16 replicas for control (untreated cells). To assay mitochondrial membrane potential, iPSC-neurons were incubated for 30 minutes with 100 nM tetramethylrhodamine, methyl ester (TMRM, ex=543 nm, em=593 nm) in addition to 20 μg/mL Hoechst (ex=377 nm, em=447 nm) and 1 μM calcein AM (ex=474 nm, em=536 nm). Cells were washed three times with warm phosphate buffered saline (PBS) and imaged immediately at 20× magnification on an imageXpress Micro confocal at 37° C. and 5% CO₂. Cell identification and quantification of TMRM intensity was automated using Molecular Devices MetaXpress software, and TMRM signal was normalized to the total cell stain calcein AM.

Following imaging, cells were immediately fixed with 4% paraformaldehyde for 30 minutes at room temperature, washed three times with PBS, permeabilized with 0.2% Triton X-100 for 30 minutes, blocked with Odyssey Blocking Buffer for 60 minutes, and incubated overnight at 4° C. with primary antibodies, neurofilament (1:1000, Synaptic Systems 171 002) and LB509 (1:100, Abcam ab27766). Cells were washed 3× with 0.1% Tween-20 in PBS and incubated with Alexa Fluor Goat Anti-Rabbit 790 (1:1000) and Goat Anti-Mouse 647 (1:1000) for 1 hour at RT, before washing 3× with 0.1% Tween-20 in PBS and imaging with Sapphire Biomolecular Imager. Neurofilament heavy chain signal intensity was normalized to fixed Hoechst signal measured with Spectra Gemini plate reader (ex=350 nm, em=461 nM), using Hoechst 33342 nucleic acid stain from Invitrogen according to the manufacturer's instructions. Neurofilaments (NF) are located mostly in axons and indicate roughly the content of neurites. If neurodegeneration is halted or reversed, NF signal increases because it correlates with the number and/or length of neurites in the well. (96)

### Results

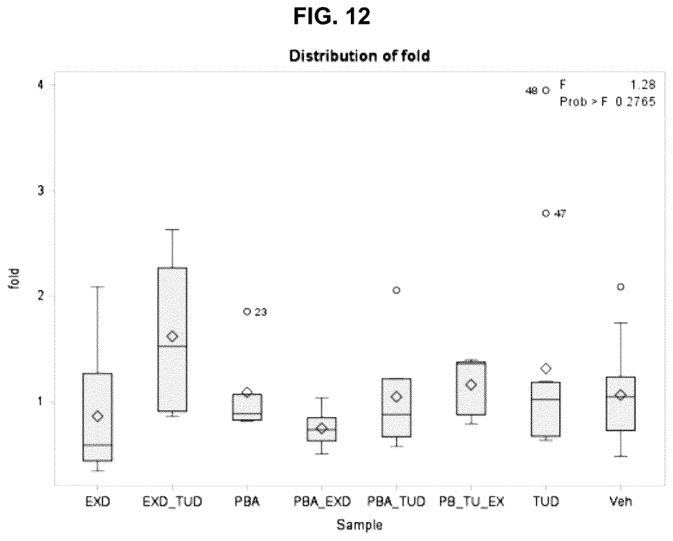

Results are shown in FIG. 12 and tabulated in Table 14. The EXD plus TUDCA combination showed higher neurofilament Heavy Chain signal intensity over EXD and TUDCA individually. Specifically, the level was 88% higher than EXD, 21% higher than TUDCA and 52% higher than the untreated cells (p<0.05). Other combinations did not show statistically significant differences from control.

TABLE 14
Neurofilament Heavy Chain signal intensity

| Obs | Sample | n | mean | Std Dev |
|---|---|---|---|---|
| 1 | EXD | 12 | 0.85955 | 0.17358 |
| 2 | EXD_TUD | 6 | 1.6165 | 0.30472 |
| 3 | PBA | 6 | 1.08812 | 0.19624 |
| 4 | PBA_EXD | 6 | 0.7455 | 0.07873 |
| 5 | PBA_TUD | 6 | 1.04423 | 0.22174 |
| 6 | PB_TU_EX | 6 | 1.1582 | 0.13435 |
| 7 | TUD | 12 | 1.31287 | 0.29185 |
| 8 | Veh | 18 | 1.06085 | 0.12421 |

### Evaluation of Effect of the Drug Combinations on Neuroinflammation Using Human Microglia Cell Line

In addition to the studies discussed above assessing activity of combinations of drugs on MPP+-induced neurodegeneration toxicity on human dopaminergic neurons as well as on a human iPSC cell line harboring a triplication mutation in alpha-synuclein gene, and measuring neuronal growth, neurite branching and neurofilament heavy chain, we also assessed combinations of drugs in reducing lipopolysaccharide (LPS, from E. coli)-induced neuroinflammation in human microglia cells. Accordingly, a study was conducted to evaluate the anti-neuroinflammatory effects of sodium phenylbutyrate (PBA), Tauroursodeoxycholic acid (TUDCA) and Exendin (EXD) in different combinations by measuring their effect on pro-inflammatory cytokines secretion following LPS and ATP stimulation of human brain microglia cells. As noted in reference (98) FIG. 1, neuroinflammation is a key pathology in synucleinopathies, so success in reducing neuroinflammation would demonstrate the value of this invention.

Lipopolysaccharides are important outer membrane components of gram-negative bacteria such as E. coli and Salmonella. They are large amphipathic glycoconjugates that typically consist of a lipid domain (hydrophobic) attached to a core oligosaccharide and a distal polysaccharide. LPS binds to Toll-like receptor 4 (TLR4) and activates microglia(98) which in turn increases secretion of pro-inflammatory cytokines such as interleukin-6 (IL-6), tumor necrosis factor-α (TNF-α), and CXCL1/GROα. Two compounds were used as positive controls in this study: TAK-242 (Resartovid), a TLR4 inhibitor, obtained from Invivogen and MCC950, a potent and specific inhibitor of the NLRP3 (NOD-like receptor pyrin domain-containing protein 3, cryopyrin, or NALP3) inflammasome (obtained from Invivogen).

### Study Design

The drugs studied are listed in Table 10.

TABLE 10
Drugs studied in LPS/ATP induced neuroinflammation

| | | | Final Vehicle |
|---|---|---|---|
| | | Testing | nature and |
| Compound | Source | concentration | amount |
| Sodium | Bio-Techne | 500, 100 μM | 0.5% H2O |
| Phenylbutyrate | (Tocris) | | |
| Tauroursodeoxycholic | Sigma | 200, 40 μM | 0.08% H2O |
| acid | (Avanti) | | |
| Exenatide | Sigma | 100, 20 nM | Culture |
| | (Aldrich) | | medium |

### Reagents

Control and Vehicle. The culture medium was BrainPhys™ Neuronal Medium, supplied by Stemcell Technologies, www.stemcell.com. Vehicles were culture medium alone, 0.5% and 0.08% H₂O in culture medium.

Lipopolysaccharide (LPS) (from E. coli 0111:B4; Invivogen reference tlrl-eblps) was prepared at a stock solution of 5 mg/mL in 100% water then diluted in culture medium.

Adenosine 5′-triphosphate (ATP) disodium salt (Invivogen; reference tlrl-atpl) was prepared at a stock solution of 725 mM in 100% water then diluted in culture medium.

### Reference Substances

TAK-242, a toll-like receptor 4 (TLR4)-specific signalling inhibitor (Resartovid, Invivogen Reference N^{o} S7455) was prepared at a stock solution of 2.76 mM in 100% DMSO and then diluted in culture medium. TAK-242 (resatorvid), a small-molecule-specific inhibitor of Toll-like receptor (TLR) 4 signaling, inhibits the production of lipopolysaccharide (LPS)-induced inflammatory and proteolytic pathways(99).

MCC950, a Nod-like receptor protein 3 (NLRP3) inhibitor (Invivogen Reference N^{o} inh-mcc) was prepared at a stock solution of 20 mM in 100% DMSO and then diluted in culture medium. MCC950 is an inhibitor of the NLRP3 inflammasome, a component of the inflammatory process(100).

Both TAK-242 and MCC950 are positive controls.

### Test Substance Preparations

Sodium Phenylbutyrate, TUDCA, and exenatide were prepared in stock solutions and diluted to the concentrations noted above.

The selected doses were chosen based on the published literature: Sodium Phenylbutyrate=500 μM(16); Exenatide=100 nM(101); TUDCA=200 μM(102), (103, 104) (104). The combination of these 3 drugs was also evaluated for dose response at a 20% dose (100 μM, 20 nM and 40 μM correspondingly).

### Study Cells

Microglia cells were obtained from an iCell® Microglia kit, 01279 (Catalogue N^{o} R1131) from FUJIFILM Cellular Dynamics. We followed the manufacturer's protocol for activation of the microglial cells with LPS/ATP to subsequently measure the effect of the study drugs on pro-inflammatory cytokines IL-6, TNF-α and CXCL1/GROα.

### Experimental Procedure

All cell culture procedures were performed in aseptic conditions, under a laminar flow hood. Microglia were thawed and cultured following the provider's instructions. The microglia were plated at 50,000 cells per well of a Corning Primaria 96 well plate (Ref N^{o} 353872) in 100 μL of growth medium. Cells were incubated at 37° C./5% CO₂ in a humidified cell culture incubator.

Three days after cell plating, the test or reference substances (25 μL of solutions at 5×) were applied 1 hour before LPS and ATP stimulation.

One hour later, 12.5 μL of test or reference substances were added again at 2× concentrated followed by 12.5 μL of LPS at 12× concentration. Fifty microliters of ATP were introduced at 4× concentration, 5 h 30 min after incubation with LPS.

Six hours after LPS incubation, the supernatant of each well was collected and centrifugated at 1000 g for 10 minutes to remove the cells and debris.

Fifty microliters of supernatant from NT and LPS+ATP-treated wells were used to determine cytokine secretion (IL-6, TNF-α, CXCL1/GROα) by Luminex platform according to the provider's instructions. Samples were diluted prior to the assay to fit within the standard range if needed. Three wells were tested per each condition (Table 11). Cytokines levels in the supernatant were expressed in pg/mL. The data was analyzed using a One-Way ANOVA followed by Bonferroni test.

TABLE 11
Experimental Combinations for
Neuroinflammation Study Conditions

| | | | | Reference | |
|---|---|---|---|---|---|
| Compound | 1 | 2 | 3 | substances | Reagents |
| Control for | 0.5% | 0.08% | Medium | | Untreated |
| #1 to #9 | H2O | H2O | | | cells |
| Control for | 0.5% | 0.08% | Medium | | LPS (100 |
| #1 to #9 | H2O | H2O | | | ng/mL) + |
| | | | | | ATP (5 mM) |
| #1 | PBA | 0.08% | Medium | | LPS (100 |
| | (500 μM) | H2O | | | ng/mL) + |
| | | | | | ATP (5 mM) |
| #2 | 0.5% | TUDCA | Medium | | LPS (100 |
| | H2O | (200 μM) | | | ng/mL) + |
| | | | | | ATP (5 mM) |
| #3 | 0.5% | 0.08% | EXD | | LPS (100 |
| | H2O | H2O | (100 nM) | | ng/mL) + |
| | | | | | ATP (5 mM) |
| #4 | PBA | 0.08% | EXD | | LPS (100 |
| | (500 μM) | H2O | (100 nM) | | ng/mL) + |
| | | | | | ATP (5 mM) |
| #5 | PBA | TUDCA | Medium | | LPS (100 |
| | (500 μM) | (200 μM) | | | ng/mL) + |
| | | | | | ATP (5 mM) |
| #6 | 0.5% | TUDCA | EXD | | LPS (100 |
| | H2O | (200 μM) | (100 nM) | | ng/mL) + |
| | | | | | ATP (5 mM) |
| #7 | PBA | TUDCA | EXD | | LPS (100 |
| | (500 μM) | (200 μM) | (100 nM) | | ng/mL) + |
| | | | | | ATP (5 mM) |
| #8 | PBA | TUDCA | EXD | | LPS (100 |
| | (100 μM) | (40 μM) | (20 nM) | | ng/mL) + |
| | | | | | ATP (5 mM) |
| #9 | PBA | 0.08% | EXD | | LPS (100 |
| | (100 μM) | H2O | (20 nM) | | ng/mL) + |
| | | | | | ATP (5 mM) |
| Positive | | | | TAK-242 | LPS (100 |
| control | | | | (2 μM) | ng/mL) + |
| | | | | | ATP (5 mM) |
| Positive | | | | MCC950 | LPS (100 |
| control | | | | (10 μM) | ng/mL) + |
| | | | | | ATP (5 mM) |

TABLE 12
Tabulation of Study Conditions

| Control/NT | Non-treated cells |
|---|---|
| Control/LPS/ATP | Activated cells |
| #1 | PBA (500 μM) |
| #2 | TUDCA (200 μM) |
| #3 | EXD (100 nM) |
| #4 | PBA (500 μM) EXD (100 nM) |
| #5 | PBA (500 μM) TUDCA (200 μM) |
| #6 | TUDCA (200 μM) EXD (100 nM) |
| #7 | PBA (500 μM) TUDCA (200 μM) |
| | EXD (100 nM) |
| #8 | PBA (100 μM) TUDCA (40 μM) |
| | EXD (20 nM) |
| #9 | PBA (100 μM) EXD (20 nM) |
| TAK-242 (2 μM)/ | Positive control |
| LPS/ATP | |
| MCC950 (10 μM)/ | Positive control |
| LPS/ATP | |

### Results

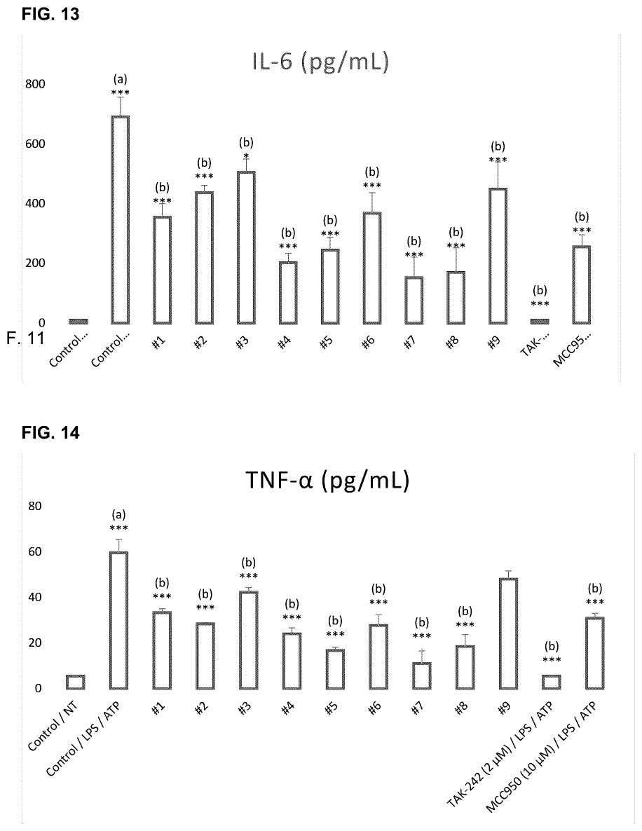

TABLE 13.1
Inhibition of IL-6 mediated inflammation
in microglial cells. (plotted in FIG. 13)

| | Repli- | Repli- | Repli- | |
|---|---|---|---|---|
| Treatment | cate 1 | cate 2 | cate 3 | Mean ± S.D. |
| Control/NT | <10.47 | <10.47 | <10.47 | 10.47 ± 0.00 |
| Control/LPS/ATP | 761.03 | 628.90 | 686.39 | 692.11 ± 66.25 |
| #1 | 396.23 | 363.94 | 305.53 | 355.23 ± 45.97 |
| #2 | 410.97 | 442.87 | 459.60 | 437.81 ± 24.71 |
| #3 | 458.31 | 509.44 | 549.19 | 505.65 ± 45.56 |
| #4 | 173.06 | 201.13 | 235.63 | 203.27 ± 31.34 |
| #5 | 195.94 | 271.31 | 269.04 | 245.43 ± 42.87 |
| #6 | 416.59 | 399.70 | 289.38 | 368.56 ± 69.09 |
| #7 | 220.79 | 154.17 | 82.65 | 152.54 ± 69.08 |
| #8 | 243.92 | 186.93 | 81.05 | 170.63 ± 82.65 |
| #9 | 432.55 | 548.35 | 369.19 | 450.03 ± 90.85 |
| TAK-242 (2 μM) | <10.47 | <10.47 | <10.47 | 10.47 ± 0.00 |
| MCC950 (10 μM) | 253.54 | 297.91 | 218.00 | 256.48 ± 40.04 |

TABLE 13.2
Inhibition of TNF-α mediated neuroinflammation
in microglial cells (plotted in FIG. 14).

| | Replicate | Replicate | Replicate | |
|---|---|---|---|---|
| Treatment | 1 | 2 | 3 | Mean ± S.D. |
| Control/NT | <5.63 | <5.63 | <5.63 | 5.63 ± 0.00 |
| Control/LPS/ATP | 53.21 | 61.30 | 64.70 | 59.74 ± 5.90 |
| #1 | 32.67 | 35.47 | 32.24 | 33.46 ± 1.75 |
| #2 | 29.01 | 27.92 | 28.79 | 28.57 ± 0.58 |
| #3 | 43.39 | 39.97 | 43.82 | 42.39 ± 2.11 |
| #4 | 21.85 | 23.59 | 27.06 | 24.17 ± 2.65 |
| #5 | 15.27 | 17.91 | 17.47 | 16.88 ± 1.41 |
| #6 | 32.02 | 28.79 | 22.72 | 27.84 ± 4.72 |
| #7 | 16.59 | 11.27 | <5.63 | 11.16 ± 5.48 |
| #8 | 23.15 | 19.88 | 12.83 | 18.62 ± 5.27 |
| #9 | 46.39 | 52.36 | 45.75 | 48.17 ± 3.65 |
| TAK-242 (2 μM)/ | <5.63 | <5.63 | <5.63 | 5.63 ± 0.00 |
| LPS/ATP | | | | |
| MCC950 (10 μM)/ | 29.44 | 29.87 | 33.53 | 30.95 ± 2.25 |
| LPS/ATP | | | | |

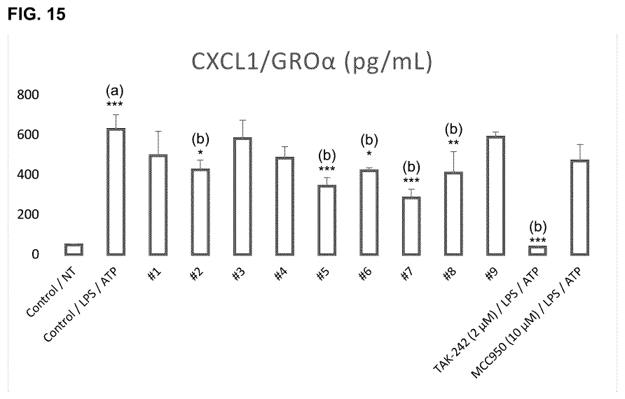

TABLE 13.3
Inhibition of CXCL1/GROα mediated neuroinflammation
in microglial cells (plotted in FIG. 15).

| | Repli- | Repli- | Repli- | |
|---|---|---|---|---|
| Treatment | cate 1 | cate 2 | cate 3 | Mean ± S.D. |
| Control/NT | 50.56 | 52.17 | 45.24 | 49.32 ± 3.63 |
| Control/LPS/ATP | 668.57 | 672.54 | 543.08 | 628.06 ± 73.62 |
| #1 | 627.29 | 383.60 | 476.49 | 495.79 ± 122.99 |
| #2 | 447.19 | 368.84 | 458.86 | 424.96 ± 48.95 |
| #3 | 658.79 | 480.28 | 606.43 | 581.83 ± 91.76 |
| #4 | 478.60 | 428.97 | 544.17 | 483.91 ± 57.78 |
| #5 | 296.66 | 378.94 | 353.94 | 343.18 ± 42.18 |
| #6 | 404.76 | 435.79 | 419.92 | 420.16 ± 15.52 |
| #7 | 325.33 | 289.90 | 238.40 | 284.54 ± 43.71 |
| #8 | 503.84 | 431.45 | 291.69 | 408.99 ± 107.84 |
| #9 | 578.48 | 570.73 | 618.42 | 589.21 ± 2 5.59 |
| TAK-242 (2 μM)/ | 35.76 | 32.96 | 44.55 | 37.76 ± 6.05 |
| LPS/ATP | | | | |
| MCC950 (10 μM)/ | 375.70 | 499.15 | 533.31 | 469.39 ± 82.91 |
| LPS/ATP | | | | |

### DISCUSSION

In FIGS. 13-15, shorter bars show a greater anti-inflammatory effect.

Control—Inflammatory Cytokines: In the presence of LPS and ATP, the concentrations of IL-6, TNF-α, and GROα were significantly increased, as compared with non-treated conditions (IL-6: 529.71±108.13 pg/mL versus 10.47±0.00 pg/mL, p<0.001; TNF-α: 49.64±11.28 pg/mL versus 5.63±0.00 pg/mL, p<0.001; and GROα: 625.26±95.84 pg/mL versus 46.09±11.38 pg/mL, p<0.001).

Condition 1: PBA at 500 μM (#1) in presence of LPS and ATP significantly decreased the concentrations of IL-6 by 49% (p<0.001) and TNF-α by 44% (p<0.001), as compared with 0.58% distilled water in presence of LPS and ATP.

Condition 2: TUDCA at 200 μM (#2) in presence of LPS and ATP significantly decreased the concentrations of IL-6 by 37% (p<0.001), TNF-α by 52% (p<0.001) and to a lesser extent, GROα by 32% (p<0.05) as compared with 0.58% distilled water in presence of LPS and ATP.

Condition 3: EXD at 100 nM (#3) in presence of LPS and ATP decreased the concentrations of IL-6 by 27% (p<0.05) and TNF-α by 29% (p<0.001), as compared with 0.58% distilled water in presence of LPS and ATP.

Condition 4: PBA at 500 UM and EXD at 100 nM (#4) in presence of LPS and ATP decreased the concentrations of IL-6 by 71% (p<0.001), TNF-α by 60% (p<0.001) as compared with 0.58% distilled water in presence of LPS and ATP. The combination of the 2 treatments also significantly decreased the concentrations of IL-6 (by 60%) and TNF-α (by 43%), as compared with EXD at 100 nM alone in presence of LPS and ATP (both p<0.001).

Condition 5: PBA at 500 UM and TUDCA at 200 UM (#5) in presence of LPS and ATP significantly decreased the concentrations of IL-6 by 65% (p<0.001), TNF-α by 72% (p<0.001) and GROα by 45% (p<0.001), as compared with 0.58% distilled water in presence of LPS and ATP. The combination of the 2 treatments also significantly decreased the concentrations of IL-6 by 44%, as compared with TUDCA at 200 UM alone in presence of LPS and ATP and the concentration of TNF-α by 50%, as compared with PBA at 500 UM alone in presence of LPS and ATP (p<0.01 for each cytokine).

Condition 6: TUDCA at 200 UM and EXD at 100 nM (#6) in presence of LPS and ATP significantly decreased the concentrations of IL-6 by 47% (p<0.001), TNF-α by 53% (p<0.001) and to a lesser extent GROα by 33% (p<0.05), as compared with 0.58% distilled water in presence of LPS and ATP. The combination of the 2 treatments also significantly decreased the concentration of TNF-α by 34%, as compared with EXD at 100 nM alone in presence of LPS and ATP (p<0.01).

Condition 7: PBA at 500 μM, TUDCA at 200 UM and EXD at 100 nM (#7) significantly decreased the concentrations of IL-6 by 78% (p<0.001), TNF-α by 81% (p<0.001) and GROα by 55% (p<0.001), as compared with 0.58% distilled water in presence of LPS and ATP. The combination of the 3 treatments also significantly decreased the concentrations of IL-6 by 57% and TNF-α by 67%, as compared with PBA at 500 UM alone (p<0.01 and p<0.001, respectively), 65% and 61% vs TUDCA at 200 UM alone (p<0.001 for both cytokines) and 70% and 74% vs EXD at 100 nM alone (p<0.001 for both cytokines) in presence of LPS and ATP. It also significantly decreased the concentration of GROα, as compared with PBA at 500 UM alone by 43% (p<0.05) and as compared with EXD at 100 nM alone by 51% (p<0.001) in presence of LPS and ATP.

Condition 8: Lower doses of PBA at 100 μM, TUDCA at 40 UM and EXD at 20 nM (#8) decreased the concentrations of IL-6 (p<0.001), TNF-α (p<0.001) and GROα (p<0.01), as compared with 0.58% distilled water in presence of LPS and ATP to lesser extent.

Condition 9: Lower doses of PBA at 100 UM and EXD at 20 UM (#9) in presence of LPS and ATP decreased the concentration of IL-6 (p<0.001), as compared with 0.58% distilled water in presence of LPS and ATP to lesser extent.

Additional combination remarks: PBA at 500 M, TUDCA at 200 UM and EXD at 100 nM (#7) significantly decreased the concentrations of IL-6 by 30%, TNF-α by 58% (p<0.05) and GROα by 41% (p<0.05), as compared with a combination of PBA at 500 UM and EXD at 100 nM (#4); and decreased the concentrations of IL-6 by 42% (p<0.05), TNF-α by 40% (p<0.05) and GROα by 17%, as compared with a combination of PBA at 500 UM and TUDCA at 200 μM (#5), and decreased the concentrations of IL-6 by 61% (p<0.001), TNF-α by 63% (p<0.001) and GROα by 33% (p<0.01), as compared with a combination of TUDCA at 200 UM and EXD at 100 nM (#6).

Positive control TAK-242, a TLR4 inhibitor at 2 μM in presence of LPS and ATP significantly decreased the concentrations of IL-6 (p<0.001), TNF-α (p<0.001), IL-1B (p<0.001) and GROα (p<0.001), as compared with 0.58% distilled water in presence of LPS and ATP.

Another positive control MCC-950, a NLRP3 inhibitor at 10 μM in presence of LPS and ATP significantly decreased the concentrations of IL-6 (p<0.001), TNF-α (p<0.001) and IL-1B (p<0.001), as compared with 0.58% distilled water in presence of LPS and ATP.

### CONCLUSIONS

Anti-inflammatory effects of the individual drugs were observed with PBA at 500 UM, TUDCA at 200 M and EXD at 100 nM. These effects were unexpectedly potentiated when the treatments were applied in different combinations. The most impressive, unexpected results were seen with a triple combination of PBA at 500 μM, TUDCA at 200 UM and EXD at 20 nM (#7) which was superior to all single and all double combinations and statistically significantly decreased levels of all three cytokines studied reducing the activation of the microglial cells, (FIG. 8, 9, 10). A combination of PBA and EXD (#4) also produced unexpected significant combinatory effect comparing to the individual drugs alone (PBA and EXD).

The effects were also observed with the combination of the three treatments at lower doses, i.e. PBA at 100 μM, TUDCA at 40 UM and EXD at 20 nM but were slightly lower suggesting dose response effect.

Because of the significance of neuroinflammation in dopaminergic neurodegeneration (FIG. 1), these results demonstrate promise in the treatment of synucleinopathic diseases.

Overall, this proves that the enhanced combinatory effect of the drugs is observed in two independent processes (dopaminergic degeneration and neuroinflammation) affecting two different cell lines (neurons and microglia) involved in neurodegenerative cascade seen in all alpha-synucleinopathies as shown in FIG. 1.

### OVERALL CONCLUSION

Various combinations of the above agents targeting different disease mechanisms simultaneously have shown substantially improved protective activity in in-vitro models and are expected to have clinical utility in slowing progression of neurodegenerative disorders and to allow these drugs to be used at lower doses to minimize potential side effects. We have shown that PBA and exenatide; TUDCA, PBA and exenatide, TUDCA and exenatide, as well as PBA, exenatide and deferiprone used in combination in a preclinical in-vitro model of Parkinson's Disease exerted a synergistic neuroprotective effect superior to each of the drugs individually. This is likely due to the combined effect of these drugs on misfolded protein (α-synuclein) accumulation, neuroinflammation, ROS formation and mitochondrial function of the human dopaminergic and microglial iPSC cells used in this experiment. The same effect can be extrapolated to other ND's including AD, ALS, HD, PSP, FTD and CBD as discussed above.

Abbreviations

| | |
|---|---|
| ALS | Amyotrophic Lateral Sclerosis |
| AD | Alzheimer's disease |
| Aβ | Amyloid β protein |
| c-Abl | cluster-Abelson tyrosine kinase |
| CBD | Corticobasal Degeneration |
| CTGF | Connective tissue growth factor |
| DPR | dipeptide repeat proteins |
| ER Stress | Endoplasmic reticulum stress |
| FTD | Frontotemporal dementia |
| Htt | huntingtin protein |
| HD | Huntington's disease |
| LPS | Lipopolysaccharide |
| MCC950 | NLRP3 inhibitor |
| MPP+ | 1-methyl-4-phenylpyridinium |
| ND | Neurodegenerative disorders |
| PCI | Pan Caspase Inhibitor |
| PD | Parkinson's disease |
| polyQ | polyglutamine |
| PSP | Progressive Supranuclear Palsy |
| ROS | Reactive oxygen species |
| SOD1 | superoxide dismutase 1 |
| TAK-242 | toll-like receptor 4 (TLR4) inhibitor (resartovid) |
| TDP43 | Tar DNA binding protein-43 |
| VCP | valosin-containing protein |

### REFERENCES

(1) Dugger, B. N.; Dickson, D. W. Pathology of Neurodegenerative Diseases. Cold Spring Harb Perspect Biol 2017, 9 (7). DOI: 10.1101/cshperspect.a028035.
(2) Ahmed, R. M.; Devenney, E. M.; Irish, M.; Ittner, A.; Naismith, S.; Ittner, L. M.; Rohrer, J. D.; Halliday, G. M.; Eisen, A.; Hodges, J. R.; Kiernan, M. C. Neuronal network disintegration: common pathways linking neurodegenerative diseases. J Neurol Neurosurg Psychiatry 2016, 87 (11), 1234-1241. DOI: 10.1136/jnnp-2014-308350.
(3) Wang, J.; Gu, B. J.; Masters, C. L.; Wang, Y. J. A systemic view of Alzheimer disease—insights from amyloid-beta metabolism beyond the brain. Nat Rev Neurol 2017, 13 (11), 703. DOI: 10.1038/nrneurol.2017.147.
(4) Chen, G. F.; Xu, T. H.; Yan, Y.; Zhou, Y. R.; Jiang, Y.; Melcher, K.; Xu, H. E. Amyloid beta: structure, biology and structure-based therapeutic development. Acta Pharmacol Sin 2017, 38 (9), 1205-1235. DOI: 10.1038/aps.2017.28.
(5) Parakh, S.; Atkin, J. D. Protein folding alterations in amyotrophic lateral sclerosis. Brain Res 2016, 1648 (Pt B), 633-649. DOI: 10.1016/j.brainres.2016.04.010.
(6) Tan, J. M.; Wong, E. S.; Lim, K. L. Protein misfolding and aggregation in Parkinson's disease. Antioxid Redox Signal 2009, 11 (9), 2119-2134. DOI: 10.1089/ARS.2009.2490.
(7) Shacham, T.; Sharma, N.; Lederkremer, G. Z. Protein Misfolding and ER Stress in Huntington's Disease. Front Mol Biosci 2019, 6, 20. DOI: 10.3389/fmolb.2019.00020.
(8) Herczenik, E.; Gebbink, M. F. Molecular and cellular aspects of protein misfolding and disease. FASEB J 2008, 22 (7), 2115-2133. DOI: 10.1096/fj.07-099671.
(9) Sweeney, P.; Park, H.; Baumann, M.; Dunlop, J.; Frydman, J.; Kopito, R.; McCampbell, A.; Leblanc, G.; Venkateswaran, A.; Nurmi, A.; Hodgson, R. Protein misfolding in neurodegenerative diseases: implications and strategies. Transl Neurodegener 2017, 6, 6. DOI: 10.1186/s40035-017-0077-5.
(10) Pedersen, J. T.; Heegaard, N. H. Analysis of protein aggregation in neurodegenerative disease. Anal Chem 2013, 85 (9), 4215-4227. DOI: 10.1021/ac400023c.
(11) Pham, C. G.; Bubici, C.; Zazzeroni, F.; Papa, S.; Jones, J.; Alvarez, K.; Jayawardena, S.; De Smaele, E.; Cong, R.; Beaumont, C.; et al. Ferritin Heavy Chain Upregulation by NF-κB Inhibits TNFα-Induced Apoptosis by Suppressing Reactive Oxygen Species. Cell 2004, 119 (4), 529-542. DOI: 10.1016/j.cell.2004.10.017.
(12) Dias, V.; Junn, E.; Mouradian, M. M. The role of oxidative stress in Parkinson's disease. Journal of Parkinson's disease 2013, 3 (4), 461-491. DOI: 10.3233/JPD-130230 PubMed.
(13) Haining, R.; Achat-Mendes, C. Neuromelanin, one of the most overlooked molecules in modern medicine, is not a spectator. Neural Regeneration Research 2017, 12 (3), 372. DOI: 10.4103/1673-5374.202928.
(14) Gerlach, M.; Double, K. L.; Ben-Shachar, D.; Zecca, L.; Youdim, M. B. H.; Riederer, P. Neuromelanin and its interaction with iron as a potential risk factor for dopaminergic neurodegeneration underlying Parkinson's disease. Neurotoxicity Research 2003, 5 (1-2), 35-43. DOI: 10.1007/bf03033371.
(15) Viceconte, N.; Burguillos, M. A.; Herrera, A. J.; De Pablos, R. M.; Joseph, B.; Venero, J. L. Neuromelanin activates proinflammatory microglia through a caspase-8-dependent mechanism. Journal of Neuroinflammation 2015, 12 (1), 5. DOI: 10.1186/s12974-014-0228-x.
(16) Roy, A.; Ghosh, A.; Jana, A.; Liu, X.; Brahmachari, S.; Gendelman, H. E.; Pahan, K. Sodium phenylbutyrate controls neuroinflammatory and antioxidant activities and protects dopaminergic neurons in mouse models of Parkinson's disease. PLOS One 2012, 7 (6), e38113. DOI: 10.1371/journal.pone.0038113.
(17) Ono, K.; Ikemoto, M.; Kawarabayashi, T.; Ikeda, M.; Nishinakagawa, T.; Hosokawa, M.; Shoji, M.; Takahashi, M.; Nakashima, M. A chemical chaperone, sodium 4-phenylbutyric acid, attenuates the pathogenic potency in human alpha-synuclein A30P+A53T transgenic mice. Parkinsonism Relat Disord 2009, 15 (9), 649-654. DOI: 10.1016/j.parkreldis.2009.03.002.
(18) Ricobaraza, A.; Cuadrado-Tejedor, M.; Perez-Mediavilla, A.; Frechilla, D.; Del Rio, J.; Garcia-Osta, A. Phenylbutyrate ameliorates cognitive deficit and reduces tau pathology in an Alzheimer's disease mouse model. Neuropsychopharmacology 2009, 34 (7), 1721-1732. DOI: 10.1038/npp.2008.229.
(19) Wiley, J. C.; Meabon, J. S.; Frankowski, H.; Smith, E. A.; Schecterson, L. C.; Bothwell, M.; Ladiges, W. C. Phenylbutyric acid rescues endoplasmic reticulum stress-induced suppression of APP proteolysis and prevents apoptosis in neuronal cells. PLOS One 2010, 5 (2), e9135. DOI: 10.1371/journal.pone.0009135.
(20) Bondulich, M. K.; Guo, T.; Meehan, C.; Manion, J.; Rodriguez Martin, T.; Mitchell, J. C.; Hortobagyi, T.; Yankova, N.; Stygelbout, V.; Brion, J. P.; et al. Tauopathy induced by low level expression of a human brain-derived tau fragment in mice is rescued by phenylbutyrate. Brain 2016, 139 (Pt 8), 2290-2306. DOI: 10.1093/brain/aww137.
(21) Zhou, W.; Bercury, K.; Cummiskey, J.; Luong, N.; Lebin, J.; Freed, C. R. Phenylbutyrate up-regulates the DJ-1 protein and protects neurons in cell culture and in animal models of Parkinson disease. J Biol Chem 2011, 286 (17), 14941-14951. DOI: 10.1074/jbc.M110.211029.
(22) Ryu, H.; Smith, K.; Camelo, S. I.; Carreras, I.; Lee, J.; Iglesias, A. H.; Dangond, F.; Cormier, K. A.; Cudkowicz, M. E.; Brown, R. H., Jr.; Ferrante, R. J. Sodium phenylbutyrate prolongs survival and regulates expression of anti-apoptotic genes in transgenic amyotrophic lateral sclerosis mice. J Neurochem 2005, 93 (5), 1087-1098. DOI: 10.1111/j.1471-4159.2005.03077.x.
(23) Del Signore, S. J.; Amante, D. J.; Kim, J.; Stack, E. C.; Goodrich, S.; Cormier, K.; Smith, K.; Cudkowicz, M. E.; Ferrante, R. J. Combined riluzole and sodium phenylbutyrate therapy in transgenic amyotrophic lateral sclerosis mice. Amyotroph Lateral Scler 2009, 10 (2), 85-94. DOI: 10.1080/17482960802226148.
(24) Cudkowicz, M. E.; Andres, P. L.; Macdonald, S. A.; Bedlack, R. S.; Choudry, R.; Brown, R. H., Jr.; Zhang, H.; Schoenfeld, D. A.; Shefner, J.; Matson, S.; et al. Phase 2 study of sodium phenylbutyrate in ALS. Amyotroph Lateral Scler 2009, 10 (2), 99-106. DOI: 10.1080/17482960802320487.
(25) Paganoni, S.; Cudkowicz, M. E. Sodium Phenylbutyrate-Taurursodiol for ALS. Reply. N Engl J Med 2020, 383 (23), 2294. DOI: 10.1056/NEJMc2030710.
(26) Gardian, G.; Browne, S. E.; Choi, D. K.; Klivenyi, P.; Gregorio, J.; Kubilus, J. K.; Ryu, H.; Langley, B.; Ratan, R. R.; Ferrante, R. J.; Beal, M. F. Neuroprotective effects of phenylbutyrate in the N171-82Q transgenic mouse model of Huntington's disease. J Biol Chem 2005, 280 (1), 556-563. DOI: 10.1074/jbc.M410210200.
(27) Hogarth, P.; Lovrecic, L.; Krainc, D. Sodium phenylbutyrate in Huntington's disease: a dose-finding study. Mov Disord 2007, 22 (13), 1962-1964. DOI: 10.1002/mds.21632.
(28) Truog, P. 4-Phenylbutyric Acid Controlled-Release Formulations for Therapeutic Use. US 2006/0045912 A1.
(29) Cortez, L.; Sim, V. The therapeutic potential of chemical chaperones in protein folding diseases. Prion 2014, 8 (2). DOI: 10.4161/pri.28938.
(30) Vang, S.; Longley, K.; Steer, C. J.; Low, W. C. The Unexpected Uses of Urso- and Tauroursodeoxycholic Acid in the Treatment of Non-liver Diseases. Glob Adv Health Med 2014, 3 (3), 58-69. DOI: 10.7453/gahmj.2014.017.
(31) Soto, C.; Pritzkow, S. Protein misfolding, aggregation, and conformational strains in neurodegenerative diseases. Nat Neurosci 2018, 21 (10), 1332-1340. DOI: 10.1038/s41593-018-0235-9.
(32) Cortez, L. M.; Campeau, J.; Norman, G.; Kalayil, M.; Van der Merwe, J.; Mckenzie, D.; Sim, V. L. Bile Acids Reduce Prion Conversion, Reduce Neuronal Loss, and Prolong Male Survival in Models of Prion Disease. J Virol 2015, 89 (15), 7660-7672. DOI: 10.1128/JVI.01165-15.
(33) Cohen, J. B.; Klee, J. COMPOSITIONS FOR IMPROVING CELL VIABILITY AND METHODS OF USE THEREOF. WO 2014/158547 A1, 2014.
(34) Lo, A. C.; Callaerts-Vegh, Z.; Nunes, A. F.; Rodrigues, C. M.; D'Hooge, R. Tauroursodeoxycholic acid (TUDCA) supplementation prevents cognitive impairment and amyloid deposition in APP/PS1 mice. Neurobiol Dis 2013, 50, 21-29. DOI: 10.1016/j.nbd.2012.09.003.
(35) Nunes, A. F.; Amaral, J. D.; Lo, A. C.; Fonseca, M. B.; Viana, R. J.; Callaerts-Vegh, Z.; D'Hooge, R.; Rodrigues, C. M. TUDCA, a bile acid, attenuates amyloid precursor protein processing and amyloid-beta deposition in APP/PS1 mice. Mol Neurobiol 2012, 45 (3), 440-454. DOI: 10.1007/s12035-012-8256-y.
(36) Kusaczuk, M. Tauroursodeoxycholate-Bile Acid with Chaperoning Activity: Molecular and Cellular Effects and Therapeutic Perspectives. Cells 2019, 8 (12). DOI: 10.3390/cells8121471.
(37) Sola, S.; Castro, R. E.; Laires, P. A.; Steer, C. J.; Rodrigues, C. M. Tauroursodeoxycholic acid prevents amyloid-beta peptide-induced neuronal death via a phosphatidylinositol 3-kinase-dependent signaling pathway. Mol Med 2003, 9 (9-12), 226-234. DOI: 10.2119/2003-00042.rodrigues.
(38) Duan, W. M.; Rodrigues, C. M.; Zhao, L. R.; Steer, C. J.; Low, W. C. Tauroursodeoxycholic acid improves the survival and function of nigral transplants in a rat model of Parkinson's disease. Cell Transplant 2002, 11 (3), 195-205.
(39) Weihe, E.; Depboylu, C.; Schutz, B.; Schafer, M. K.; Eiden, L. E. Three types of tyrosine hydroxylase-positive CNS neurons distinguished by dopa decarboxylase and VMAT2 co-expression. Cell Mol Neurobiol 2006, 26 (4-6), 659-678. DOI: 10.1007/s10571-006-9053-9.
(40) Castro-Caldas, M.; Carvalho, A. N.; Rodrigues, E.; Henderson, C. J.; Wolf, C. R.; Rodrigues, C. M.; Gama, M. J. Tauroursodeoxycholic acid prevents MPTP-induced dopaminergic cell death in a mouse model of Parkinson's disease. Mol Neurobiol 2012, 46 (2), 475-486. DOI: 10.1007/s12035-012-8295-4.
(41) Rosa, A. I.; Fonseca, I.; Nunes, M. J.; Moreira, S.; Rodrigues, E.; Carvalho, A. N.; Rodrigues, C. M. P.; Gama, M. J.; Castro-Caldas, M. Novel insights into the antioxidant role of tauroursodeoxycholic acid in experimental models of Parkinson's disease. Biochim Biophys Acta Mol Basis Dis 2017, 1863 (9), 2171-2181. DOI: 10.1016/j.bbadis.2017.06.004.
(42) Elia, A. E.; Lalli, S.; Monsurro, M. R.; Sagnelli, A.; Taiello, A. C.; Reggiori, B.; La Bella, V.; Tedeschi, G.; Albanese, A. Tauroursodeoxycholic acid in the treatment of patients with amyotrophic lateral sclerosis. Eur J Neurol 2016, 23 (1), 45-52. DOI: 10.1111/ene.12664.
(43) Keene, C. D.; Rodrigues, C. M.; Eich, T.; Linehan-Stieers, C.; Abt, A.; Kren, B. T.; Steer, C. J.; Low, W. C. A bile acid protects against motor and cognitive deficits and reduces striatal degeneration in the 3-nitropropionic acid model of Huntington's disease. Exp Neurol 2001, 171 (2), 351-360. DOI: 10.1006/exnr.2001.7755.
(44) Keene, C. D.; Rodrigues, C. M.; Eich, T.; Chhabra, M. S.; Steer, C. J.; Low, W. C. Tauroursodeoxycholic acid, a bile acid, is neuroprotective in a transgenic animal model of Huntington's disease. Proc Natl Acad Sci USA 2002, 99 (16), 10671-10676. DOI: 10.1073/pnas.162362299.
(45) Pockley, A. G. Heat shock proteins as regulators of the immune response. Lancet 2003, 362 (9382), 469-476. DOI: 10.1016/S0140-6736(03)14075-5. Hartl, F. U. Molecular chaperones in cellular protein folding. Nature 1996, 381 (6583), 571-579. DOI: 10.1038/381571a0 From NLM.
(46) Wilhelmus, M. M.; Boelens, W. C.; Otte-Holler, I.; Kamps, B.; de Waal, R. M.; Verbeek, M. M. Small heat shock proteins inhibit amyloid-beta protein aggregation and cerebrovascular amyloid-beta protein toxicity. Brain Res 2006, 1089 (1), 67-78. DOI: 10.1016/j.brainres.2006.03.058.
(47) Dou, F.; Netzer, W. J.; Tanemura, K.; Li, F.; Hartl, F. U.; Takashima, A.; Gouras, G. K.; Greengard, P.; Xu, H. Chaperones increase association of tau protein with microtubules. Proc Natl Acad Sci USA 2003, 100 (2), 721-726. DOI: 10.1073/pnas.242720499.
(48) Auluck, P. K.; Chan, H. Y.; Trojanowski, J. Q.; Lee, V. M.; Bonini, N. M. Chaperone suppression of alpha-synuclein toxicity in a Drosophila model for Parkinson's disease. Science 2002, 295 (5556), 865-868. DOI: 10.1126/science.1067389.
(49) Klucken, J.; Shin, Y.; Masliah, E.; Hyman, B. T.; McLean, P. J. Hsp70 Reduces alpha-Synuclein Aggregation and Toxicity. J Biol Chem 2004, 279 (24), 25497-25502. DOI: 10.1074/jbc.M400255200.
(50) Dedmon, M. M.; Christodoulou, J.; Wilson, M. R.; Dobson, C. M. Heat shock protein 70 inhibits alpha-synuclein fibril formation via preferential binding to prefibrillar species. J Biol Chem 2005, 280 (15), 14733-14740. DOI: 10.1074/jbc.M413024200.
(51) Kalmar, B.; Novoselov, S.; Gray, A.; Cheetham, M. E.; Margulis, B.; Greensmith, L. Late stage treatment with arimoclomol delays disease progression and prevents protein aggregation in the SOD1 mouse model of ALS. J Neurochem 2008, 107 (2), 339-350. DOI: 10.1111/j.1471-4159.2008.05595.x.
(52) Benatar, M.; Wuu, J.; Andersen, P. M.; Atassi, N.; David, W.; Cudkowicz, M.; Schoenfeld, D. Randomized, double-blind, placebo-controlled trial of arimoclomol in rapidly progressive SOD1 ALS. Neurology 2018, 90 (7), e565-e574. DOI: 10.1212/WNL.0000000000004960.
(53) Carmichael, J.; Chatellier, J.; Woolfson, A.; Milstein, C.; Fersht, A. R.; Rubinsztein, D. C. Bacterial and yeast chaperones reduce both aggregate formation and cell death in mammalian cell models of Huntington's disease. Proc Natl Acad Sci USA 2000, 97 (17), 9701-9705. DOI: 10.1073/pnas.170280697.
(54) Hughes, R. E.; Olson, J. M. Therapeutic opportunities in polyglutamine disease. Nat Med 2001, 7 (4), 419-423. DOI: 10.1038/86486.
(55) Krobitsch, S.; Lindquist, S. Aggregation of huntingtin in yeast varies with the length of the polyglutamine expansion and the expression of chaperone proteins. Proc Natl Acad Sci USA 2000, 97 (4), 1589-1594. DOI: 10.1073/pnas.97.4.1589.
(56) Cudkowicz, M. E.; Shefner, J. M.; Simpson, E.; Grasso, D.; Yu, H.; Zhang, H.; Shui, A.; Schoenfeld, D.; Brown, R. H.; Wieland, S.; et al. Arimoclomol at dosages up to 300 mg/day is well tolerated and safe in amyotrophic lateral sclerosis. Muscle Nerve 2008, 38 (1), 837-844. DOI: 10.1002/mus.21059.
(57) Kim, S.; Moon, M.; Park, S. Exendin-4 protects dopaminergic neurons by inhibition of microglial activation and matrix metalloproteinase-3 expression in an animal model of Parkinson's disease. J Endocrinol 2009, 202 (3), 431-439. DOI: 10.1677/JOE-09-0132.
(58) Chen, S.; Yu, S. J.; Li, Y.; Lecca, D.; Glotfelty, E.; Kim, H. K.; Choi, H. I.; Hoffer, B. J.; Greig, N. H.; Kim, D. S.; Wang, Y. Post-treatment with PT302, a long-acting Exendin-4 sustained release formulation, reduces dopaminergic neurodegeneration in a 6-Hydroxydopamine rat model of Parkinson's disease. Sci Rep 2018, 8 (1), 10722. DOI: 10.1038/s41598-018-28449-z.
(59) Zhang, L.; Zhang, L.; Li, L.; Holscher, C. Semaglutide is Neuroprotective and Reduces alpha-Synuclein Levels in the Chronic MPTP Mouse Model of Parkinson's Disease. J Parkinsons Dis 2019, 9 (1), 157-171. DOI: 10.3233/JPD-181503.
(60) Igoillo-Esteve, M.; Oliveira, A. F.; Cosentino, C.; Fantuzzi, F.; Demarez, C.; Toivonen, S.; Hu, A.; Chintawar, S.; Lopes, M.; Pachera, N.; et al. Exenatide induces frataxin expression and improves mitochondrial function in Friedreich ataxia. JCI Insight 2020, 5 (2). DOI: 10.1172/jci.insight. 134221. Chang, G.; Liu, J.; Qin, S.; Jiang, Y.; Zhang, P.; Yu, H.; Lu, K.; Zhang, N.; Cao, L.; Wang, Y.; et al. Cardioprotection by exenatide: A novel mechanism via improving mitochondrial function involving the GLP-1 receptor/cAMP/PKA pathway. Int J Mol Med 2018, 41 (3), 1693-1703. DOI: 10.3892/ijmm.2017.3318.
(61) Athauda, D.; Maclagan, K.; Skene, S. S.; Bajwa-Joseph, M.; Letchford, D.; Chowdhury, K.; Hibbert, S.; Budnik, N.; Zampedri, L.; Dickson, J.; et al. Exenatide once weekly versus placebo in Parkinson's disease: a randomised, double-blind, placebo-controlled trial. Lancet 2017, 390 (10103), 1664-1675. DOI: 10.1016/S0140-6736(17)31585-4.
(62) Methods and compositions for oral administration of exenatide. U.S. Pat. No. 10,350,162B2.
(63) Yun, S. P.; Kam, T. I.; Panicker, N.; Kim, S.; Oh, Y.; Park, J. S.; Kwon, S. H.; Park, Y. J.; Karuppagounder, S. S.; Park, H.; et al. Block of A1 astrocyte conversion by microglia is neuroprotective in models of Parkinson's disease. Nat Med 2018, 24 (7), 931-938. DOI: 10.1038/s41591-018-0051-5 From NLM.
(64) Mullins, R. J.; Mustapic, M.; Chia, C. W.; Carlson, O.; Gulyani, S.; Tran, J.; Li, Y.; Mattson, M. P.; Resnick, S.; Egan, J. M.; et al. A Pilot Study of Exenatide Actions in Alzheimer's Disease. Curr Alzheimer Res 2019, 16 (8), 741-752. DOI: 10.2174/1567205016666190913155950.
(65) Li, Y.; Chigurupati, S.; Holloway, H. W.; Mughal, M.; Tweedie, D.; Bruestle, D. A.; Mattson, M. P.; Wang, Y.; Harvey, B. K.; Ray, B.; et al. Exendin-4 ameliorates motor neuron degeneration in cellular and animal models of amyotrophic lateral sclerosis. PLOS One 2012, 7 (2), e32008. DOI: 10.1371/journal.pone.0032008.
(66) Martin, B.; Golden, E.; Carlson, O. D.; Pistell, P.; Zhou, J.; Kim, W.; Frank, B. P.; Thomas, S.; Chadwick, W. A.; Greig, N. H.; et al. Exendin-4 improves glycemic control, ameliorates brain and pancreatic pathologies, and extends survival in a mouse model of Huntington's disease. Diabetes 2009, 58 (2), 318-328. DOI: 10.2337/db08-0799.
(67) Cabantchik, Z. I.; Munnich, A.; Youdim, M. B.; Devos, D. Regional siderosis: a new challenge for iron chelation therapy. Front Pharmacol 2013, 4, 167. DOI: 10.3389/fphar.2013.00167.
(68) Devos, D.; Moreau, C.; Devedjian, J. C.; Kluza, J.; Petrault, M.; Laloux, C.; Jonneaux, A.; Ryckewaert, G.; Garcon, G.; Rouaix, N.; et al. Targeting chelatable iron as a therapeutic modality in Parkinson's disease. Antioxid Redox Signal 2014, 21 (2), 195-210. DOI: 10.1089/ars.2013.5593.
(69) Rogers, J. T.; Bush, A. I.; Cho, H. H.; Smith, D. H.; Thomson, A. M.; Friedlich, A. L.; Lahiri, D. K.; Leedman, P. J.; Huang, X.; Cahill, C. M. Iron and the translation of the amyloid precursor protein (APP) and ferritin mRNAs: riboregulation against neural oxidative damage in Alzheimer's disease. Biochem Soc Trans 2008, 36 (Pt 6), 1282-1287. DOI: 10.1042/BST0361282.
(70) Lovell, M. A.; Robertson, J. D.; Teesdale, W. J.; Campbell, J. L.; Markesbery, W. R. Copper, iron and zinc in Alzheimer's disease senile plaques. J Neurol Sci 1998, 158 (1), 47-52. DOI: 10.1016/s0022-510x(98)00092-6.
(71) Rao, S. S.; Portbury, S. D.; Lago, L.; Bush, A. I.; Adlard, P. A. The Iron Chelator Deferiprone Improves the Phenotype in a Mouse Model of Tauopathy. J Alzheimers Dis 2020, 78 (4), 1783. DOI: 10.3233/JAD-209009.
(72) Rao, S. S.; Lago, L.; Volitakis, I.; Shukla, J. J.; McColl, G.; Finkelstein, D. I.; Adlard, P. A. Deferiprone Treatment in Aged Transgenic Tau Mice Improves Y-Maze Performance and Alters Tau Pathology. Neurotherapeutics 2021, 18 (2), 1081-1094. DOI: 10.1007/s13311-020-00972-w.
(73) Ritchie, C. W.; Bush, A. I.; Mackinnon, A.; Macfarlane, S.; Mastwyk, M.; MacGregor, L.; Kiers, L.; Cherny, R.; Li, Q. X.; Tammer, A.; et al. Metal-protein attenuation with iodochlorhydroxyquin (clioquinol) targeting Abeta amyloid deposition and toxicity in Alzheimer disease: a pilot phase 2 clinical trial. Arch Neurol 2003, 60 (12), 1685-1691. DOI: 10.1001/archneur.60.12.1685.
(74) Bharathi; Rao, K. S. Thermodynamics imprinting reveals differential binding of metals to alpha-synuclein: relevance to Parkinson's disease. Biochem Biophys Res Commun 2007, 359 (1), 115-120. DOI: 10.1016/j.bbrc.2007.05.060.
(75) Castellani, R. J.; Siedlak, S. L.; Perry, G.; Smith, M. A. Sequestration of iron by Lewy bodies in Parkinson's disease. Acta Neuropathol 2000, 100 (2), 111-114. DOI: 10.1007/s004010050001.
(76) Lovejoy, D. B.; Guillemin, G. J. The potential for transition metal-mediated neurodegeneration in amyotrophic lateral sclerosis. Front Aging Neurosci 2014, 6, 173. DOI: 10.3389/fnagi.2014.00173.
(77) Yu, J.; Qi, F.; Wang, N.; Gao, P.; Dai, S.; Lu, Y.; Su, Q.; Du, Y.; Che, F. Increased iron level in motor cortex of amyotrophic lateral sclerosis patients: an in vivo MR study. Amyotroph Lateral Scler Frontotemporal Degener 2014, 15 (5-6), 357-361. DOI: 10.3109/21678421.2014.906618.
(78) Sheelakumari, R.; Madhusoodanan, M.; Radhakrishnan, A.; Ranjith, G.; Thomas, B. A Potential Biomarker in Amyotrophic Lateral Sclerosis: Can Assessment of Brain Iron Deposition with SWI and Corticospinal Tract Degeneration with DTI Help? AJNR Am J Neuroradiol 2016, 37 (2), 252-258. DOI: 10.3174/ajnr.A4524.
(79) Moreau, C.; Danel, V.; Devedjian, J. C.; Grolez, G.; Timmerman, K.; Laloux, C.; Petrault, M.; Gouel, F.; Jonneaux, A.; Dutheil, M.; et al. Could Conservative Iron Chelation Lead to Neuroprotection in Amyotrophic Lateral Sclerosis? Antioxid Redox Signal 2018, 29 (8), 742-748. DOI: 10.1089/ars.2017.7493.
(80) Niu, L.; Ye, C.; Sun, Y.; Peng, T.; Yang, S.; Wang, W.; Li, H. Mutant huntingtin induces iron overload via up-regulating IRP1 in Huntington's disease. Cell Biosci 2018, 8, 41. DOI: 10.1186/s13578-018-0239-x.
(81) Agrawal, S.; Fox, J.; Thyagarajan, B.; Fox, J. H. Brain mitochondrial iron accumulates in Huntington's disease, mediates mitochondrial dysfunction, and can be removed pharmacologically. Free Radic Biol Med 2018, 120, 317-329. DOI: 10.1016/j.freeradbiomed.2018.04.002.
(82) MOUSSA, C. Treating neural disease with tyrosine kinase inhibitors. WO2013/166295 A1, 2013.
(83) Werner, M. H.; Olanow, C. W. Parkinson's Disease Modification Through Abl Kinase Inhibition: An Opportunity. Movement Disorders 2022, 37 (1), 6-15. DOI: https://doi.org/10.1002/mds.28858.
(84) Karuppagounder, S. S.; Brahmachari, S.; Lee, Y.; Dawson, V. L.; Dawson, T. M.; Ko, H. S. The c-Abl inhibitor, nilotinib, protects dopaminergic neurons in a preclinical animal model of Parkinson's disease. Sci Rep 2014, 4, 4874. DOI: 10.1038/srep04874 From NLM.
(85) Pagan, F. L.; Hebron, M. L.; Wilmarth, B.; Torres-Yaghi, Y.; Lawler, A.; Mundel, E. E.; Yusuf, N.; Starr, N. J.; Anjum, M.; Arellano, J.; et al. Nilotinib Effects on Safety, Tolerability, and Potential Biomarkers in Parkinson Disease: A Phase 2 Randomized Clinical Trial. JAMA Neurol 2020, 77 (3), 309-317. DOI: 10.1001/jamaneurol.2019.4200.
(86) Lonskaya, I.; Hebron, M. L.; Selby, S. T.; Turner, R. S.; Moussa, C. E. Nilotinib and bosutinib modulate pre-plaque alterations of blood immune markers and neuro-inflammation in Alzheimer's disease models. Neuroscience 2015, 304, 316-327. DOI: 10.1016/j.neuroscience.2015.07.070.
(87) La Barbera, L.; Vedele, F.; Nobili, A.; Krashia, P.; Spoleti, E.; Latagliata, E. C.; Cutuli, D.; Cauzzi, E.; Marino, R.; Viscomi, M. T.; et al. Nilotinib restores memory function by preventing dopaminergic neuron degeneration in a mouse model of Alzheimer's Disease. Prog Neurobiol 2021, 202, 102031. DOI: 10.1016/j.pneurobio.2021.102031.
(88) Turner, R. S.; Hebron, M. L.; Lawler, A.; Mundel, E. E.; Yusuf, N.; Starr, J. N.; Anjum, M.; Pagan, F.; Torres-Yaghi, Y.; Shi, W.; et al. Nilotinib Effects on Safety, Tolerability, and Biomarkers in Alzheimer's Disease. Ann Neurol 2020, 88 (1), 183-194. DOI: 10.1002/ana.25775.
(89) Hebron, M. L.; Lonskaya, I.; Moussa, C. E. Nilotinib reverses loss of dopamine neurons and improves motor behavior via autophagic degradation of alpha-synuclein in Parkinson's disease models. Hum Mol Genet 2013, 22 (16), 3315-3328. DOI: 10.1093/hmg/ddt192.
(90) Pagan, F. L.; Wilmarth, B.; Torres-Yaghi, Y.; Hebron, M. L.; Mulki, S.; Ferrante, D.; Matar, S.; Ahn, J.; Moussa, C. Long-Term Safety and Clinical Effects of Nilotinib in Parkinson's Disease. Mov Disord 2021, 36 (3), 740-749. DOI: 10.1002/mds.28389.
(91) Katsumata, R.; Ishigaki, S.; Katsuno, M.; Kawai, K.; Sone, J.; Huang, Z.; Adachi, H.; Tanaka, F.; Urano, F.; Sobue, G. c-Abl inhibition delays motor neuron degeneration in the G93A mouse, an animal model of amyotrophic lateral sclerosis. PLOS One 2012, 7 (9), e46185. DOI: 10.1371/journal.pone.0046185.
(92) Rojas, F.; Gonzalez, D.; Cortes, N.; Ampuero, E.; Hernandez, D. E.; Fritz, E.; Abarzua, S.; Martinez, A.; Elorza, A. A.; Alvarez, A.; et al. Reactive oxygen species trigger motoneuron death in non-cell-autonomous models of ALS through activation of c-Abl signaling. Front Cell Neurosci 2015, 9, 203. DOI: 10.3389/fncel.2015.00203.
(93) Corrochano, S.; Renna, M.; Carter, S.; Chrobot, N.; Kent, R.; Stewart, M.; Cooper, J.; Brown, S. D.; Rubinsztein, D. C.; Acevedo-Arozena, A. alpha-Synuclein levels modulate Huntington's disease in mice. Hum Mol Genet 2012, 21 (3), 485-494. DOI: 10.1093/hmg/ddr477.
(94) Kalivendi, S. V.; Kotamraju, S.; Cunningham, S.; Shang, T.; Hillard, C. J.; Kalyanaraman, B. 1-Methyl-4-phenylpyridinium (MPP+)-induced apoptosis and mitochondrial oxidant generation: role of transferrin-receptor-dependent iron and hydrogen peroxide. Biochem J 2003, 371 (Pt 1), 151-164. DOI: 10.1042/bj20021525 From NLM.
(95) ApexBio. https://www.apexbt.com/q-vd-oph-hydrate.html (accessed Sep. 17, 2022).
(96) Zala, D.; Benchoua, A.; Brouillet, E.; Perrin, V.; Gaillard, M. C.; Zurn, A. D.; Aebischer, P.; Déglon, N. Progressive and selective striatal degeneration in primary neuronal cultures using lentiviral vector coding for a mutant huntingtin fragment. Neurobiol Dis 2005, 20 (3), 785-798. DOI: 10.1016/j.nbd.2005.05.017 From NLM.
(97) Yuan, A.; Nixon, R. A. Neurofilament Proteins as Biomarkers to Monitor Neurological Diseases and the Efficacy of Therapies. Frontiers in Neuroscience 2021, 15, Review. DOI: 10.3389/fnins.2021.689938.
(98) Bassil, R.; Shields, K.; Granger, K.; Zein, I.; Ng, S.; Chih, B. Improved modeling of human AD with an automated culturing platform for iPSC neurons, astrocytes and microglia. Nat Commun 2021, 12 (1), 5220. DOI: 10.1038/s41467-021-25344-6 From NLM Medline.
(99) Ono, Y.; Maejima, Y.; Saito, M.; Sakamoto, K.; Horita, S.; Shimomura, K.; Inoue, S.; Kotani, J. TAK-242, a specific inhibitor of Toll-like receptor 4 signalling, prevents endotoxemia-induced skeletal muscle wasting in mice. Sci Rep 2020, 10 (1), 694. DOI: 10.1038/s41598-020-57714-3 From NLM Medline.
(100) Coll, R. C.; Robertson, A. A.; Chae, J. J.; Higgins, S. C.; Munoz-Planillo, R.; Inserra, M. C.; Vetter, I.; Dungan, L. S.; Monks, B. G.; Stutz, A.; et al. A small-molecule inhibitor of the NLRP3 inflammasome for the treatment of inflammatory diseases. Nat Med 2015, 21 (3), 248-255. DOI: 10.1038/nm.3806 From NLM Medline.
(101) Gullo, F.; Ceriani, M.; D'Aloia, A.; Wanke, E.; Constanti, A.; Costa, B.; Lecchi, M. Plant Polyphenols and Exendin-4 Prevent Hyperactivity and TNF-alpha Release in LPS-Treated In vitro Neuron/Astrocyte/Microglial Networks. Front Neurosci 2017, 11, 500. DOI: 10.3389/fnins.2017.00500 From NLM PubMed-not-MEDLINE.
(102) Romero-Ramirez, L.; Garcia-Rama, C.; Wu, S.; Mey, J. Bile acids attenuate PKM2 pathway activation in proinflammatory microglia. Sci Rep 2022, 12 (1), 1459. DOI: 10.1038/s41598-022-05408-3 From NLM Medline.
(103) Yanguas-Casas, N.; Barreda-Manso, M. A.; Nieto-Sampedro, M.; Romero-Ramirez, L. TUDCA: An Agonist of the Bile Acid Receptor GPBAR1/TGR5 With Anti-Inflammatory Effects in Microglial Cells. J Cell Physiol 2017, 232 (8), 2231-2245. DOI: 10.1002/jcp.25742 From NLM Medline.
(104) Kim, S. J.; Ko, W. K.; Jo, M. J.; Arai, Y.; Choi, H.; Kumar, H.; Han, I. B.; Sohn, S. Anti-inflammatory effect of Tauroursodeoxycholic acid in RAW 264.7 macrophages, Bone marrow-derived macrophages, BV2 microglial cells, and spinal cord injury. Sci Rep 2018, 8 (1), 3176. DOI: 10.1038/s41598-018-21621-5 From NLM Medline.

1. A method of treating a neurodegenerative disorder or preventing a neurodegenerative disorder selected from Alzheimer's disease (AD), Parkinson's disease (PD), Amyotrophic Lateral Sclerosis (ALS), Huntington's disease (HD), Progressive Supranuclear Palsy (PSP), Frontotemporal dementia (FTD) and Corticobasal Degeneration (CBD), wherein the method consists of the administration of a composition consisting of a combination of two or more drugs selected from at least two distinct pharmacological classes of drugs, wherein the distinct classes of drugs consist of a sodium 4-phenylbutyrate chemical chaperone class of drugs, a bile acid class of drugs, a glucagon-like-peptide-1 agonist (GLP-1) class of drugs, an iron chelator class of drugs, and a cluster-Abelson (c-Abl) tyrosine kinase inhibitor class of drugs.

2. The method of claim 1, wherein the method consists of a composition consisting of a combination of at least three drugs selected from three of the distinct pharmacological classes.

3. The method of claim 1, wherein the pharmacological classes of drugs consist of:
a. a sodium 4-phenylbutyrate chemical chaperone class of drugs;
b. a bile acid class of drugs comprising one or more of tauroursodeoxycholic acid (TUDCA), ursodeoxycholic acid (UDCA) and deoxycholic acid (DCA);
c. wherein the GLP-1 class of drugs comprises one or more of exenatide, ORMD-0901, dulaglutide, semaglutide, tirzepatide, liraglutide, NLY01, or lixisenatide;
d. wherein the iron chelator class of drugs comprises a drug selected from deferiprone (DFP), deferoxamine (DFO), desferrioxamine, deferasirox, clioquinol, tetrahydrosalen, 5,7-Dichloro-2-[(dimethylamino)methyl]quinolin-8-ol (PBT2), (N,N,N,N-Tetrakis(2-pyridylmethyl)-ethylenedi-amine) (TPEN), 1,10-phenanthroline (PHEN), 1,2-hydroxypyridinone (1,2-HOPO), clioquinol; 5-[N-methyl-N-propargylaminomethyl]-8-hydroxyquinoline dihydrochloride (M30); M31; M32; -[4-(2-hydroxyethyl)piperazine-1-ylmethyl]-quinoline-8-ol] (VK28), HLA16, HLA20, M32, M10, SIH-B, BSIH, pyridoxal isonicotinoyl hydrazine (PIH); 2-pyridylcarboxaldehyde isonicotinoyl hydrazine (PCIH), H2NPH, and H2PPH;
e. wherein the c-Abl tyrosine kinase inhibitor class of drugs comprises a drug selected from nilotinib, radotinib, vodobatinib (K0706), bafetinib, imatinib, dasatinib, bosutinib, ponatinib, rebastinib, tozasertib, danusertib, and IKT-148009.

4. The method of claim 1 wherein the composition consists of sodium phenylbutyrate (PBA) and exenatide (EXD).

5. The method of claim 1 wherein the composition consists of PBA, tauroursodeoxycholic acid (TUDCA), and EXD.

6. The method of claim 1 wherein the composition consists of dasatinib (DAS) and TUDCA.

7. The method of claim 1 wherein the composition consists of EXD and TUDCA.

8. The method of claim 1 wherein the composition consists of EXD, DAS, and TUDCA.

9. The method of claim 1 wherein the composition consists of PBA, EXD, and deferiprone (DFP).

10. The method of claim 1 wherein the composition consists of PBA, DAS, and TUDCA.

11. The method of claim 1 wherein the composition consists of PBA and DAS.

12. The method of claim 1 wherein the composition consists of EXD and DAS.

13. The method of claim 1 wherein the composition consists of PBA, EXD, and DAS.

14. The method of claim 1 wherein the composition consists of PBA, EXD, TUDCA, and DFP.

15. The method of claim 1, wherein sodium phenylbutyrate (PBA) is provided as an extended-release formulation.

16. A method of treating a neurodegenerative disorder or preventing a neurodegenerative disorder associated with misfolding of proteins selected from the group consisting of tau proteins (AD, PSP, CBD), amyloid (AD), alpha-synuclein (PD), superoxide dismutase 1 (SOD1) (ALS), Tar DNA binding protein-43 (TDP43) (ALS), Ubiquilin-2 (ALS), p62 (ALS), valosin-containing protein (VCP) (ALS), mutant huntingtin protein (mHtt) (HD), and dipeptide repeat (DPR) (ALS) proteins, wherein the method consists of the administration of a composition consisting of a combination of two or more drugs selected from at least two distinct pharmacological classes of drugs, wherein the distinct classes of drugs consist of a sodium 4-phenylbutyrate chemical chaperone class of drugs, a bile acid class of drugs, a glucagon-like-peptide-1 agonist (GLP-1) class of drugs, an iron chelator class of drugs, and a cluster-Abelson (c-Abl) tyrosine kinase inhibitor class of drugs.
